SEQUENCE LISTING (98 sequences)

SEQ ID NO: 1 - protein, 36 aa, Artificial Sequence
  source: /note=“Description of Artificial Sequence Synthetic polypeptide”
  VARIANT (1)..(1): /replace=“R” or “N” or “S” or “D” or “L” or “Q” or “I”
  VARIANT (5)..(5): /replace=“H” or “P” or “S”
  VARIANT (8)..(8): /replace=“Y”
  VARIANT (9)..(9): /replace=“S”
  VARIANT (12)..(12): /replace=“H” or “Q”
  VARIANT (15)..(15): /replace=“S”
  VARIANT (16)..(16): /replace=“V” or “Y”
  VARIANT (18)..(18): /replace=“E” or “R”
  VARIANT (19)..(19): /replace=“I” or “N”
  VARIANT (22)..(22): /replace=“E” or “R”
  VARIANT (23)..(23): /replace=“E”
  VARIANT (25)..(25): /replace=“R”
  VARIANT (26)..(26): /replace=“T” or “Y”
  VARIANT (27)..(27): /replace=“L” or “R”
  VARIANT (28)..(28): /replace=“N”
  VARIANT (29)..(29): /replace=“L” or “R”
  VARIANT (30)..(30): /replace=“D” or “E” or “F” or “G” or “I” or “K” or “L” or “P” or “Q” or “R” or “S” or “T” or “Y”
  SITE (1)..(36): /note=“Variant residues given in the sequence have no preference with respect to those in the annotations for variant positions”
  SITE (31)..(36): /note=“This region may encompass one of the following sequences ′QAPA′ or ′QAPK′ or ′QAPR′ or ′QAPAVD′ or ′QAPKVD′ or ′QAPRVD′”
  source: /note=“See specification as filed for detailed description of substitutions and preferred embodiments”
AQRRGFIARLREDPEFSAHLLADAKQDADAXXXXXX

SEQ ID NO: 2 - protein, 4 aa, Artificial Sequence
  source: /note=“Description of Artificial Sequence Synthetic peptide”
  VARIANT (4)..(4): /replace=“K” or “R”
  SITE (1)..(4): /note=“Variant residues given in the sequence have no preference with respect to those in the annotations for variant positions”
QAPA

SEQ ID NO: 3 - protein, 6 aa, Artificial Sequence
  source: /note=“Description of Artificial Sequence Synthetic peptide”
  VARIANT (4)..(4): /replace=“K” or “R”
  SITE (1)..(6): /note=“Variant residues given in the sequence have no preference with respect to those in the annotations for variant positions”
QAPAVD

SEQ ID NO: 4 - protein, 24 aa, Artificial Sequence
  source: /note=“Description of Artificial Sequence Synthetic peptide”
VDAKFDKELEEARAEIERLPNLTE

SEQ ID NO: 5 - protein, 24 aa, Artificial Sequence
  source: /note=“Description of Artificial Sequence Synthetic peptide”
VDAKFDKELEEIRAEIERLPNLTE

SEQ ID NO: 6 - protein, 5 aa, Artificial Sequence
  source: /note=“Description of Artificial Sequence Synthetic peptide”
MAQGT

SEQ ID NO: 7 - protein, 6 aa, Artificial Sequence
  source: /note=“Description of Artificial Sequence Synthetic peptide”
QAPKVD

SEQ ID NO: 8 - protein, 6 aa, Artificial Sequence
  source: /note=“Description of Artificial Sequence Synthetic peptide”
QAPRVD

SEQ ID NO: 9 - protein, 36 aa, Artificial Sequence
  source: /note=“Description of Artificial Sequence Synthetic polypeptide”
  VARIANT (4)..(4): /replace=“K” or “R”
  SITE (1)..(36): /note=“Variant residues given in the sequence have no preference with respect to those in the annotations for variant positions”
  SITE (5)..(36): /note=“This region may encompass one of the following sequences ′VDGQAGQGGGSGLNDIFEAQKIEWHEHHHHHH′ or ′GQAGQGGGSGLNDIFEAQKIEW ′HEHHHHHH′ or ′VDGLNDIFEAQKIEWHEHHHHHH′ or ′GLNDIFEAQKIEWHEHHH HHH′”
  source: /note=“See specification as filed for detailed description of substitutions and preferred embodiments”
QAPAXXXXXXXXXXXXXXXXXXXXXXXXXXXXXXXX

SEQ ID NO: 10 - protein, 32 aa, Artificial Sequence
  source: /note=“Description of Artificial Sequence Synthetic polypeptide”
VDGQAGQGGGSGLNDIFEAQKIEWHEHHHHHH

SEQ ID NO: 11 - protein, 30 aa, Artificial Sequence
  source: /note=“Description of Artificial Sequence Synthetic polypeptide”
GQAGQGGGSGLNDIFEAQKIEWHEHHHHHH

SEQ ID NO: 12 - protein, 23 aa, Artificial Sequence
  source: /note=“Description of Artificial Sequence Synthetic peptide”
VDGLNDIFEAQKIEWHEHHHHHH

SEQ ID NO: 13 - protein, 21 aa, Artificial Sequence
  source: /note=“Description of Artificial Sequence Synthetic peptide”
GLNDIFEAQKIEWHEHHHHHH

SEQ ID NO: 14 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDIQAPKGQAGQGGGSGLNDIFEAQKIEWHEHHHHHH

SEQ ID NO: 15 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAHLLADAKYRNDTQAPKGQAGQGGGSGLNDIFEAQKIEWHEHHHHHH

SEQ ID NO: 16 - protein, 93 aa, Artificial Sequence
  source: /note=“Description of Artificial Sequence Synthetic polypeptide”
MAQGTVDAKFDKELEEARAEIERLPNLTENQRRSFIYRLRQDPSFSAILLADAKYRNDIQAPKGQAGQGGGSGLNDIFEAQKIEWHEHHHHHH

SEQ ID NO: 17 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSPSAILLADAKYRNDIQAPKGQAGQGGGSGLNDIFEAQKIEWHEHHHHHH

SEQ ID NO: 18 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSVSAILLADAKYRNDIQAPKGQAGQGGGSGLNDIFEAQKIEWHEHHHHHH

SEQ ID NO: 19 - protein, 93 aa, Artificial Sequence
  source: /note=“Description of Artificial Sequence Synthetic polypeptide”
MAQGTVDAKFDKELEEARAEIERLPNLTESQRRSFIYRLRQDPSFSAILLADAKYRNDIQAPKGQAGQGGGSGLNDIFEAQKIEWHEHHHHHH

SEQ ID NO: 20 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSYSAILLADAKYRNDIQAPKGQAGQGGGSGLNDIFEAQKIEWHEHHHHHH

SEQ ID NO: 21 - protein, 93 aa, Artificial Sequence
  source: /note=“Description of Artificial Sequence Synthetic polypeptide”
MAQGTVDAKFDKELEEARAEIERLPNLTERQRRSFIYRLREDPSFSAILLADAKYRNDSQAPKGQAGQGGGSGLNDIFEAQKIEWHEHHHHHH

SEQ ID NO: 22 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDQQAPKGQAGQGGGSGLNDIFEAQKIEWHEHHHHHH

SEQ ID NO: 23 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDAQAPKGQAGQGGGSGLNDIFEAQKIEWHEHHHHHH

SEQ ID NO: 24 - protein, 93 aa, Artificial Sequence
  source: /note=“Description of Artificial Sequence Synthetic polypeptide”
MAQGTVDAKFDKELEEARAEIERLPNLTEAQRRSFIYRLRQDPSFSAILLADAKYRNDFQAPKGQAGQGGGSGLNDIFEAQKIEWHEHHHHHH

SEQ ID NO: 25 - protein, 93 aa, Artificial Sequence
  source: /note=“Description of Artificial Sequence Synthetic polypeptide”
MAQGTVDAKFDKELEEARAEIERLPNLTEDQRRSFIYRLRQDPSFSAILLADAKYRNDIQAPKGQAGQGGGSGLNDIFEAQKIEWHEHHHHHH

SEQ ID NO: 26 - protein, 93 aa, Artificial Sequence
  source: /note=“Description of Artificial Sequence Synthetic polypeptide”
MAQGTVDAKFDKELEEARAEIERLPNLTERQRRPFIYRLRQDPSFSAILLADAKYRNDIQAPKGQAGQGGGSGLNDIFEAQKIEWHEHHHHHH

SEQ ID NO: 27 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYLNDIQAPKGQAGQGGGSGLNDIFEAQKIEWHEHHHHHH

SEQ ID NO: 28 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDDQAPKGQAGQGGGSGLNDIFEAQKIEWHEHHHHHH

SEQ ID NO: 29 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDRQAPKGQAGQGGGSGLNDIFEAQKIEWHEHHHHHH

SEQ ID NO: 30 - protein, 93 aa, Artificial Sequence
  source: /note=“Description of Artificial Sequence Synthetic polypeptide”
MAQGTVDAKFDKELEEARAEIERLPNLTELQRRSFIYRLRQDPSFSAILLADAKYRNDIQAPKGQAGQGGGSGLNDIFEAQKIEWHEHHHHHH

SEQ ID NO: 31 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDGQAPKGQAGQGGGSGLNDIFEAQKIEWHEHHHHHH

SEQ ID NO: 32 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKTRNDIQAPKGQAGQGGGSGLNDIFEAQKIEWHEHHHHHH

SEQ ID NO: 33 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDKQAPKGQAGQGGGSGLNDIFEAQKIEWHEHHHHHH

SEQ ID NO: 34 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDYQAPKGQAGQGGGSGLNDIFEAQKIEWHEHHHHHH

SEQ ID NO: 35 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDPQAPKGQAGQGGGSGLNDIFEAQKIEWHEHHHHHH

SEQ ID NO: 36 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSANLLADAKYRNDEQAPKGQAGQGGGSGLNDIFEAQKIEWHEHHHHHH

SEQ ID NO: 37 - protein, 93 aa, Artificial Sequence
  source: /note=“Description of Artificial Sequence Synthetic polypeptide”
MAQGTVDAKFDKELEEARAEIERLPNLTERQRRHFIYRLRQDPSFSAILLADAKYRNDIQAPKGQAGQGGGSGLNDIFEAQKIEWHEHHHHHH

SEQ ID NO: 38 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKQRNDIQAPKGQAGQGGGSGLNDIFEAQKIEWHEHHHHHH

SEQ ID NO: 39 - protein, 93 aa, Artificial Sequence
  source: /note=“Description of Artificial Sequence Synthetic polypeptide”
MAQGTVDAKFDKELEEARAEIERLPNLTERQRRSFIYSLRQDPSFSAILLADAKYRNDIQAPKGQAGQGGGSGLNDIFEAQKIEWHEHHHHHH

SEQ ID NO: 40 - protein, 93 aa, Artificial Sequence
  source: /note=“Description of Artificial Sequence Synthetic polypeptide”
MAQGTVDAKFDKELEEARAEIERLPNLTERQRRSFIARLRQDPSFSAILLADAKYRNDIQAPKGQAGQGGGSGLNDIFEAQKIEWHEHHHHHH

SEQ ID NO: 41 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDSQAPKGQAGQGGGSGLNDIFEAQKIEWHEHHHHHH

SEQ ID NO: 42 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDVQAPKGQAGQGGGSGLNDIFEAQKIEWHEHHHHHH

SEQ ID NO: 43 - protein, 93 aa, Artificial Sequence
  source: /note=“Description of Artificial Sequence Synthetic polypeptide”
MAQGTVDAKFDKELEEARAEIERLPNLTEIQRRSFIYRLRQDPSFSAILLADAKYRNDIQAPKGQAGQGGGSGLNDIFEAQKIEWHEHHHHHH

SEQ ID NO: 44 - protein, 93 aa, Artificial Sequence
  source: /note=“Description of Artificial Sequence Synthetic polypeptide”
MAQGTVDAKFDKELEEARAEIERLPNLTERQRRSFIYRLRHDPSFSAILLADAKYRNDRQAPKGQAGQGGGSGLNDIFEAQKIEWHEHHHHHH

SEQ ID NO: 45 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDTQAPKGQAGQGGGSGLNDIFEAQKIEWHEHHHHHH

SEQ ID NO: 46 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSPSAILLADAKYRNDLQAPKGQAGQGGGSGLNDIFEAQKIEWHEHHHHHH

SEQ ID NO: 47 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNDLQAPKGQAGQGGGSGLNDIFEAQKIEWHEHHHHHH

SEQ ID NO: 48 - protein, 93 aa, Artificial Sequence
  source: /note=“Description of Artificial Sequence Synthetic polypeptide”
MAQGTVDAKFDKELEEARAEIERLPNLTERQRRSFIYRLRQDPEFSAILLADAKYRNDIQAPKGQAGQGGGSGLNDIFEAQKIEWHEHHHHHH

SEQ ID NO: 49 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSRILLEDAKYRNDIQAPKGQAGQGGGSGLNDIFEAQKIEWHEHHHHHH

SEQ ID NO: 50 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSEILLRDAKYRNDIQAPKGQAGQGGGSGLNDIFEAQKIEWHEHHHHHH

SEQ ID NO: 51 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRADIQAPKGQAGQGGGSGLNDIFEAQKIEWHEHHHHHH

SEQ ID NO: 52 - protein, 93 aa, Artificial Sequence
  source: /note=“Description of Artificial Sequence Synthetic polypeptide”
MAQGTVDAKFDKELEEARAEIERLPNLTERQRRSFIYRLRQDPSFSAILLADAKYRNRIQAPKGQAGQGGGSGLNDIFEAQKIEWHEHHHHHH

SEQ ID NO: 53 - protein, 61 aa, Artificial Sequence
  source: /note=“Description of Artificial Sequence Synthetic polypeptide”
MVDAKFDKELEEARAEIERLPNLTERQRRSFIYRLRQDPSFSAILLADAKYRNDIQAPKVD

SEQ ID NO: 54 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDIQAPK

SEQ ID NO: 55 - protein, 58 aa, Artificial Sequence
  source: /note=“Description of Artificial Sequence Synthetic polypeptide”
VDAKFDKELEEARAEIERLPNLTERQRRSFIYRLRQDPSFSAHLLADAKYRNDTQAPK

SEQ ID NO: 56 - protein, 58 aa, Artificial Sequence
  source: /note=“Description of Artificial Sequence Synthetic polypeptide”
VDAKFDKELEEARAEIERLPNLTENQRRSFIYRLRQDPSFSAILLADAKYRNDIQAPK

SEQ ID NO: 57 - protein, 58 aa, Artificial Sequence
  source: /note=“Description of Artificial Sequence Synthetic polypeptide”
VDAKFDKELEEARAEIERLPNLTERQRRSFIYRLRQDPSPSAILLADAKYRNDIQAPK

SEQ ID NO: 58 - protein, 58 aa, Artificial Sequence
  source: /note=“Description of Artificial Sequence Synthetic polypeptide”
VDAKFDKELEEARAEIERLPNLTERQRRSFIYRLRQDPSVSAILLADAKYRNDIQAPK

SEQ ID NO: 59 - protein, 58 aa, Artificial Sequence
  source: /note=“Description of Artificial Sequence Synthetic polypeptide”
VDAKFDKELEEARAEIERLPNLTESQRRSFIYRLRQDPSFSAILLADAKYRNDIQAPK

SEQ ID NO: 60 - protein, 58 aa, Artificial Sequence
  source: /note=“Description of Artificial Sequence Synthetic polypeptide”
VDAKFDKELEEARAEIERLPNLTERQRRSFIYRLRQDPSYSAILLADAKYRNDIQAPK

SEQ ID NO: 61 - protein, 58 aa, Artificial Sequence
  source: /note=“Description of Artificial Sequence Synthetic polypeptide”
VDAKFDKELEEARAEIERLPNLTERQRRSFIYRLREDPSFSAILLADAKYRNDSQAPK

SEQ ID NO: 62 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDQQAPK

SEQ ID NO: 63 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDAQAPK

SEQ ID NO: 64 - protein, 58 aa, Artificial Sequence
  source: /note=“Description of Artificial Sequence Synthetic polypeptide”
VDAKFDKELEEARAEIERLPNLTEAQRRSFIYRLRQDPSFSAILLADAKYRNDFQAPK

SEQ ID NO: 65 - protein, 58 aa, Artificial Sequence
  source: /note=“Description of Artificial Sequence Synthetic polypeptide”
VDAKFDKELEEARAEIERLPNLTEDQRRSFIYRLRQDPSFSAILLADAKYRNDIQAPK

SEQ ID NO: 66 - protein, 58 aa, Artificial Sequence
  source: /note=“Description of Artificial Sequence Synthetic polypeptide”
VDAKFDKELEEARAEIERLPNLTERQRRPFIYRLRQDPSFSAILLADAKYRNDIQAPK

SEQ ID NO: 67 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYLNDIQAPK

SEQ ID NO: 68 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDDQAPK

SEQ ID NO: 69 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDRQAPK

SEQ ID NO: 70 - protein, 58 aa, Artificial Sequence
  source: /note=“Description of Artificial Sequence Synthetic polypeptide”
VDAKFDKELEEARAEIERLPNLTELQRRSFIYRLRQDPSFSAILLADAKYRNDIQAPK

SEQ ID NO: 71 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDGQAPK

SEQ ID NO: 72 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKTRNDIQAPK

SEQ ID NO: 73 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDKQAPK

SEQ ID NO: 74 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDYQAPK

SEQ ID NO: 75 - protein, 57 aa, Artificial Sequence
  source: /note=“Description of Artificial Sequence Synthetic polypeptide”
DAKFDKELEEARAEIERLPNLTERQRRSFIYRLRQDPSFSAILLADAKYRNDPQAPK

SEQ ID NO: 76 - protein, 57 aa, Artificial Sequence
  source: /note=“Description of Artificial Sequence Synthetic polypeptide”
DAKFDKELEEARAEIERLPNLTERQRRSFIYRLRQDPSFSANLLADAKYRNDEQAPK

SEQ ID NO: 77 - protein, 58 aa, Artificial Sequence
  source: /note=“Description of Artificial Sequence Synthetic polypeptide”
VDAKFDKELEEARAEIERLPNLTERQRRHFIYRLRQDPSFSAILLADAKYRNDIQAPK

SEQ ID NO: 78 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKQRNDIQAPK

SEQ ID NO: 79 - protein, 58 aa, Artificial Sequence
  source: /note=“Description of Artificial Sequence Synthetic polypeptide”
VDAKFDKELEEARAEIERLPNLTERQRRSFIYSLRQDPSFSAILLADAKYRNDIQAPK

SEQ ID NO: 80 - protein, 58 aa, Artificial Sequence
  source: /note=“Description of Artificial Sequence Synthetic polypeptide”
VDAKFDKELEEARAEIERLPNLTERQRRSFIARLRQDPSFSAILLADAKYRNDIQAPK

SEQ ID NO: 81 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDSQAPK

SEQ ID NO: 82 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDVQAPK

SEQ ID NO: 83 - protein, 58 aa, Artificial Sequence
  source: /note=“Description of Artificial Sequence Synthetic polypeptide”
VDAKFDKELEEARAEIERLPNLTEIQRRSFIYRLRQDPSFSAILLADAKYRNDIQAPK

SEQ ID NO: 84 - protein, 58 aa, Artificial Sequence
  source: /note=“Description of Artificial Sequence Synthetic polypeptide”
VDAKFDKELEEARAEIERLPNLTERQRRSFIYRLRHDPSFSAILLADAKYRNDRQAPK

SEQ ID NO: 85 - protein, 58 aa, Artificial Sequence; identical to SEQ ID NO: 84
  source: /note=“Description of Artificial Sequence Synthetic polypeptide”

SEQ ID NO: 86 - protein, 58 aa, Artificial Sequence
  source: /note=“Description of Artificial Sequence Synthetic polypeptide”
VDAKFDKELEEARAEIERLPNLTERQRRSFIYRLRQDPSPSAILLADAKYRNDLQAPK

SEQ ID NO: 87 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNDLQAPK

SEQ ID NO: 88 - protein, 58 aa, Artificial Sequence
  source: /note=“Description of Artificial Sequence Synthetic polypeptide”
VDAKFDKELEEARAEIERLPNLTERQRRSFIYRLRQDPEFSAILLADAKYRNDIQAPK

SEQ ID NO: 89 - protein, 58 aa, Artificial Sequence
  source: /note=“Description of Artificial Sequence Synthetic polypeptide”
VDAKFDKELEEARAEIERLPNLTERQRRSFIYRLRQDPSFSRILLEDAKYRNDIQAPK

SEQ ID NO: 90 - protein, 57 aa, Artificial Sequence
  source: /note=“Description of Artificial Sequence Synthetic polypeptide”
DAKFDKELEEARAEIERLPNLTERQRRSFIYRLRQDPSFSEILLRDAKYRNDIQAPK

SEQ ID NO: 91 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRADIQAPK

SEQ ID NO: 92 - protein, 58 aa, Artificial Sequence
  source: /note=“Description of Artificial Sequence Synthetic polypeptide”
VDAKFDKELEEARAEIERLPNLTERQRRSFIYRLRQDPSFSAILLADAKYRNRIQAPK

SEQ ID NO: 93 - protein, 58 aa, Artificial Sequence; identical to SEQ ID NO: 54
  source: /note=“Description of Artificial Sequence Synthetic polypeptide”

SEQ ID NO: 94 - protein, 93 aa, Artificial Sequence
  source: /note=“Description of Artificial Sequence Synthetic polypeptide”
MAQGTVDAKFDKEQIEADTEIIWLPNLNLLQFKAFIKSLLDDPSQSANLLAEAKKLNDAQAPKGQAGQGGGSGLNDIFEAQKIEWHEHHHHHH

SEQ ID NO: 95 - protein, 603 aa, Artificial Sequence
  source: /note=“Description of Artificial Sequence Synthetic polypeptide”
  SITE (1)..(580): /note=“This region may encompass 2-10 repeating ′VDAKFDKELEEARAE IERLPNLTEX1QRRX2FIX3X4LRX5DPX6X7SX8X9LLX10X11AX12 X13X14X15X16X17 QAPX′ units wherein X is A, K, or R; X1 is A, R, N, S, D, L, Q or I; X2 is G, H, P or S; X3 is A or Y; X4 is R or S; X5 is E, H”
  SITE (1)..(580): /note=“CONT. FROM ABOVE or Q; X6 is E or S ; X7 is F, V or Y; X8 is A, E or R; X9 is H, I or N ; X10 is A, E or R; X11 is D or E; X12 is K or R; X13 is Q, T or Y; X14 is D, L or R; X15 is A or N; X16 is D, L or R; and X17 is A, D, E, F, G, I, K, L,”
  SITE (1)..(580): /note=“CONT. FROM ABOVEP, Q, R, S, T or Y”
  VARIANT (25)..(25): /replace=“R” or “N” or “S” or “D” or “L” or “Q” or “I”
  VARIANT (29)..(29): /replace=“H” or “P” or “S”
  VARIANT (32)..(32): /replace=“Y”
  VARIANT (33)..(33): /replace=“S”
  VARIANT (36)..(36): /replace=“H” or “Q”
  VARIANT (39)..(39): /replace=“S”
  VARIANT (40)..(40): /replace=“V” or “Y”
  VARIANT (42)..(42): /replace=“E” or “R”
  VARIANT (43)..(43): /replace=“I” or “N”
  VARIANT (46)..(46): /replace=“E” or “R”
  VARIANT (47)..(47): /replace=“E”
  VARIANT (49)..(49): /replace=“R”
  VARIANT (50)..(50): /replace=“T” or “Y”
  VARIANT (51)..(51): /replace=“L” or “R”
  VARIANT (52)..(52): /replace=“N”
  VARIANT (53)..(53): /replace=“L” or “R”
  VARIANT (54)..(54): /replace=“D” or “E” or “F” or “G” or “I” or “K” or “L” or “P” or “Q” or “R” or “S” or “T” or “Y”
  VARIANT (58)..(58): /replace=“K” or “R”
  VARIANT (83)..(83): /replace=“R” or “N” or “S” or “D” or “L” or “Q” or “I”
  VARIANT (87)..(87): /replace=“H” or “P” or “S”
  VARIANT (90)..(90): /replace=“Y”
  VARIANT (91)..(91): /replace=“S”
  VARIANT (94)..(94): /replace=“H” or “Q”
  VARIANT (97)..(97): /replace=“S”
  VARIANT (98)..(98): /replace=“V” or “Y”
  VARIANT (100)..(100): /replace=“E” or “R”
  VARIANT (101)..(101): /replace=“I” or “N”
  VARIANT (104)..(104): /replace=“E” or “R”
  VARIANT (105)..(105): /replace=“E”
  VARIANT (107)..(107): /replace=“R”
  VARIANT (108)..(108): /replace=“T” or “Y”
  VARIANT (109)..(109): /replace=“L” or “R”
  VARIANT (110)..(110): /replace=“N”
  VARIANT (111)..(111): /replace=“L” or “R”
  VARIANT (112)..(112): /replace=“D” or “E” or “F” or “G” or “I” or “K” or “L” or “P” or “Q” or “R” or “S” or “T” or “Y”
  VARIANT (116)..(116): /replace=“K” or “R”
  VARIANT (141)..(141): /replace=“R” or “N” or “S” or “D” or “L” or “Q” or “I”
  VARIANT (145)..(145): /replace=“H” or “P” or “S”
  VARIANT (148)..(148): /replace=“Y”
  VARIANT (149)..(149): /replace=“S”
  VARIANT (152)..(152): /replace=“H” or “Q”
  VARIANT (155)..(155): /replace=“S”
  VARIANT (156)..(156): /replace=“V” or “Y”
  VARIANT (158)..(158): /replace=“E” or “R”
  VARIANT (159)..(159): /replace=“I” or “N”
  VARIANT (162)..(162): /replace=“E” or “R”
  VARIANT (163)..(163): /replace=“E”
  VARIANT (165)..(165): /replace=“R”
  VARIANT (166)..(166): /replace=“T” or “Y”
  VARIANT (167)..(167): /replace=“L” or “R”
  VARIANT (168)..(168): /replace=“N”
  VARIANT (169)..(169): /replace=“L” or “R”
  VARIANT (170)..(170): /replace=“D” or “E” or “F” or “G” or “I” or “K” or “L” or “P” or “Q” or “R” or “S” or “T” or “Y”
  VARIANT (174)..(174): /replace=“K” or “R”
  VARIANT (199)..(199): /replace=“R” or “N” or “S” or “D” or “L” or “Q” or “I” FEATURE
  VARIANT (206)..(206): /replace=“Y”
  VARIANT (207)..(207): /replace=“S”
  VARIANT (210)..(210): /replace=“H” or “Q”
  VARIANT (213)..(213): /replace=“S”
  VARIANT (214)..(214): /replace=“V” or “Y”
  VARIANT (216)..(216): /replace=“E” or “R”
  VARIANT (217)..(217): /replace=“I” or “N”
  VARIANT (220)..(220): /replace=“E” or “R”
  VARIANT (221)..(221): /replace=“E”
  VARIANT (223)..(223): /replace=“R”
  VARIANT (224)..(224): /replace=“T” or “Y”
  VARIANT (225)..(225): /replace=“L” or “R”
  VARIANT (226)..(226): /replace=“N”
  VARIANT (227)..(227): /replace=“L” or “R”
  VARIANT (228)..(228): /replace=“D” or “E” or “F” or “G” or “I” or “K” or “L” or “P” or “Q” or “R” or “S” or “T” or “Y”
  VARIANT (232)..(232): /replace=“K” or “R”
  VARIANT (257)..(257): /replace=“R” or “N” or “S” or “D” or “L” or “Q” or “I”
  VARIANT (261)..(261): /replace=“H” or “P” or “S”
  VARIANT (264)..(264): /replace=“Y”
  VARIANT (265)..(265): /replace=“S”
  VARIANT (268)..(268): /replace=“H” or “Q”
  VARIANT (271)..(271): /replace=“S”
  VARIANT (272)..(272): /replace=“V” or “Y”
  VARIANT (274)..(274): /replace=“E” or “R”
  VARIANT (275)..(275): /replace=“I” or “N”
  VARIANT (278)..(278): /replace=“E” or “R”
  VARIANT (279)..(279): /replace=“E”
  VARIANT (281)..(281): /replace=“R”
  VARIANT (282)..(282): /replace=“T” or “Y”
  VARIANT (283)..(283): /replace=“L” or “R”
  VARIANT (284)..(284): /replace=“N”
  VARIANT (285)..(285): /replace=“L” or “R”
  VARIANT (286)..(286): /replace=“D” or “E” or “F” or “G” or “I” or “K” or “L” or “P” or “Q” or “R” or “S” or “T” or “Y”
  VARIANT (290)..(290): /replace=“K” or “R”
  VARIANT (315)..(315): /replace=“R” or “N” or “S” or “D” or “L” or “Q” or “I” FEATURE
  VARIANT (322)..(322): /replace=“Y”
  VARIANT (323)..(323): /replace=“S”
  VARIANT (326)..(326): /replace=“H” or “Q”
  VARIANT (329)..(329): /replace=“S”
  VARIANT (330)..(330): /replace=“V” or “Y”
  VARIANT (332)..(332): /replace=“E” or “R”
  VARIANT (333)..(333): /replace=“I” or “N”
  VARIANT (336)..(336): /replace=“E” or “R”
  VARIANT (337)..(337): /replace=“E”
  VARIANT (339)..(339): /replace=“R”
  VARIANT (340)..(340): /replace=“T” or “Y”
  VARIANT (341)..(341): /replace=“L” or “R”
  VARIANT (342)..(342): /replace=“N”
  VARIANT (343)..(343): /replace=“L” or “R”
  VARIANT (344)..(344): /replace=“D” or “E” or “F” or “G” or “I” or “K” or “L” or “P” or “Q” or “R” or “S” or “T” or “Y”
  VARIANT (348)..(348): /replace=“K” or “R”
  VARIANT (373)..(373): /replace=“R” or “N” or “S” or “D” or “L” or “Q” or “I” FEATURE
  VARIANT (380)..(380): /replace=“Y”
  VARIANT (381)..(381): /replace=“S”
  VARIANT (384)..(384): /replace=“H” or “Q”
  VARIANT (387)..(387): /replace=“S”
  VARIANT (388)..(388): /replace=“V” or “Y”
  VARIANT (390)..(390): /replace=“E” or “R”
  VARIANT (391)..(391): /replace=“I” or “N”
  VARIANT (394)..(394): /replace=“E” or “R”
  VARIANT (395)..(395): /replace=“E”
  VARIANT (397)..(397): /replace=“R”
  VARIANT (398)..(398): /replace=“T” or “Y”
  VARIANT (399)..(399): /replace=“L” or “R”
  VARIANT (400)..(400): /replace=“N”
  VARIANT (401)..(401): /replace=“L” or “R”
  VARIANT (402)..(402): /replace=“D” or “E” or “F” or “G” or “I” or “K” or “L” or “P” or “Q” or “R” or “S” or “T” or “Y”
  VARIANT (406)..(406): /replace=“K” or “R”
  VARIANT (431)..(431): /replace=“R” or “N” or “S” or “D” or “L” or “Q” or “I” FEATURE
  VARIANT (438)..(438): /replace=“Y”
  VARIANT (439)..(439): /replace=“S”
  VARIANT (442)..(442): /replace=“H” or “Q”
  VARIANT (445)..(445): /replace=“S”
  VARIANT (446)..(446): /replace=“V” or “Y”
  VARIANT (448)..(448): /replace=“E” or “R”
  VARIANT (449)..(449): /replace=“I” or “N”
  VARIANT (452)..(452): /replace=“E” or “R”
  VARIANT (453)..(453): /replace=“E”
  VARIANT (455)..(455): /replace=“R”
  VARIANT (456)..(456): /replace=“T” or “Y”
  VARIANT (457)..(457): /replace=“L” or “R”
  VARIANT (458)..(458): /replace=“N”
  VARIANT (459)..(459): /replace=“L” or “R”
  VARIANT (460)..(460): /replace=“D” or “E” or “F” or “G” or “I” or “K” or “L” or “P” or “Q” or “R” or “S” or “T” or “Y”
  VARIANT (464)..(464): /replace=“K” or “R”
  VARIANT (489)..(489): /replace=“R” or “N” or “S” or “D” or “L” or “Q” or “I” FEATURE
  VARIANT (496)..(496): /replace=“Y”
  VARIANT (497)..(497): /replace=“S”
  VARIANT (500)..(500): /replace=“H” or “Q”
  VARIANT (503)..(503): /replace=“S”
  VARIANT (504)..(504): /replace=“V” or “Y”
  VARIANT (506)..(506): /replace=“E” or “R”
  VARIANT (507)..(507): /replace=“I” or “N”
  VARIANT (510)..(510): /replace=“E” or “R”
  VARIANT (511)..(511): /replace=“E”
  VARIANT (513)..(513): /replace=“R”
  VARIANT (514)..(514): /replace=“T” or “Y”
  VARIANT (515)..(515): /replace=“L” or “R”
  VARIANT (516)..(516): /replace=“N”
  VARIANT (517)..(517): /replace=“L” or “R”
  VARIANT (518)..(518): /replace=“D” or “E” or “F” or “G” or “I” or “K” or “L” or “P” or “Q” or “R” or “S” or “T” or “Y”
  VARIANT (522)..(522): /replace=“K” or “R”
  VARIANT (547)..(547): /replace=“R” or “N” or “S” or “D” or “L” or “Q” or “I” FEATURE
  VARIANT (554)..(554): /replace=“Y”
  VARIANT (555)..(555): /replace=“S”
  VARIANT (558)..(558): /replace=“H” or “Q”
  VARIANT (561)..(561): /replace=“S”
  VARIANT (562)..(562): /replace=“V” or “Y”
  VARIANT (564)..(564): /replace=“E” or “R”
  VARIANT (565)..(565): /replace=“I” or “N”
  VARIANT (568)..(568): /replace=“E” or “R”
  VARIANT (569)..(569): /replace=“E”
  VARIANT (571)..(571): /replace=“R”
  VARIANT (572)..(572): /replace=“T” or “Y”
  VARIANT (573)..(573): /replace=“L” or “R”
  VARIANT (574)..(574): /replace=“N”
  VARIANT (575)..(575): /replace=“L” or “R”
  VARIANT (576)..(576): /replace=“D” or “E” or “F” or “G” or “I” or “K” or “L” or “P” or “Q” or “R” or “S” or “T” or “Y”
  VARIANT (580)..(580): /replace=“K” or “R”
  SITE (1)..(603): /note=“Variant residues given in the sequence have no preference with respect to those in the annotations for variant positions”
VDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDAKFDKELEEARAEIERLPNLTEAQRRGFIARLREDPEFSAHLLADAKQDADAQAPAVDGLNDIFEAQKIEWHEHHHHHH

SEQ ID NO: 96 - protein, 30 aa, Artificial Sequence
  source: /note=“Description of Artificial Sequence Synthetic polypeptide”
  VARIANT (1)..(1): /replace=“R” or “N” or “S” or “D” or “L” or “Q” or “I”
  VARIANT (5)..(5): /replace=“H” or “P” or “S”
  VARIANT (8)..(8): /replace=“Y”
  VARIANT (9)..(9): /replace=“S”
  VARIANT (12)..(12): /replace=“H” or “Q”
  VARIANT (15)..(15): /replace=“S”
  VARIANT (16)..(16): /replace=“V” or “Y”
  VARIANT (18)..(18): /replace=“E” or “R”
  VARIANT (19)..(19): /replace=“I” or “N”
  VARIANT (22)..(22): /replace=“E” or “R”
  VARIANT (23)..(23): /replace=“E”
  VARIANT (25)..(25): /replace=“R”
  VARIANT (26)..(26): /replace=“T” or “Y”
  VARIANT (27)..(27): /replace=“L” or “R”
  VARIANT (28)..(28): /replace=“N”
  VARIANT (29)..(29): /replace=“L” or “R”
  VARIANT (30)..(30): /replace=“D” or “E” or “F” or “G” or “I” or “K” or “L” or “P” or “Q” or “R” or “S” or “T” or “Y”
  SITE (1)..(30): /note=“Variant residues given in the sequence have no preference with respect to those in the annotations for variant positions”
  source: /note=“See specification as filed for detailed description of substitutions and preferred embodiments”
AQRRGFIARLREDPEFSAHLLADAKQDADA

SEQ ID NO: 97 - protein, 5 aa, Artificial Sequence
  source: /note=“Description of Artificial Sequence Synthetic peptide”
GGGGG

SEQ ID NO: 98 - protein, 8 aa, Artificial Sequence
  source: /note=“Description of Artificial Sequence Synthetic peptide”
GGGGGGGG

(19) United States
(12) Patent Application Publication
(10) Pub. No.: US 2023/0391829 A1
(43) Pub. Date: Dec. 7, 2023

(54) AAV8 AFFINITY AGENTS

(71) Applicant: Avitide LLC, Lebanon, NH (US)

(72) Inventors: Sarmistha Bhattacharya, Lebanon, NH (US); Brandon Coyle, Lebanon, NH (US); Sarah Valentini, Newport, NH (US); Kelley Kearns, Grantham, NH (US); Thomas Scanlon, Lebanon, NH (US); William Wellborn, Norwich, VT (US); Warren Kett, West Lebanon, NH (US)

(21) Appl. No.: 18/031,833

(22) PCT Filed: Oct. 13, 2021
(86) PCT No.: PCT/US21/54870

§ 371 Date: Apr. 13, 2023

Related U.S. Application Data
(60) Provisional application No. 63/091,207, filed on Oct. 13, 2020.

(51) Int. Cl. C07K 14/00; C07K 1/22; B01D 15/38

(52) U.S. Cl. CPC C07K 14/001; C07K 2319/21; B01D 15/3804; C07K 1/22

(57) ABSTRACT

Provided herein are affinity ligands that specifically interact with AAV8 capsid and/or AAV8 variant capsid, affinity agents comprising the affinity ligands, and methods of their use for binding, isolation, and/or purification of AAV8 and variants thereof.

### CROSS REFERENCE TO RELATED APPLICATIONS

This application claims the priority benefit of U.S. Provisional Application No. 63/091,207 filed on Oct. 13, 2020, the entire contents of which is incorporated by reference herein.

### FIELD OF THE DISCLOSURE

The present disclosure relates to the field of chromatography, and more specifically to novel affinity ligands and affinity agents which are suitable for use in isolation of adeno-associated virus (AAV). Thus, the disclosure encompasses affinity ligands as such, chromatography separation matrices (affinity agents) comprising an affinity ligand according to the disclosure, and a process of AAV isolation, particularly AAV serotype 8 (AAV8) isolation wherein the ligand according to the disclosure is used.

### BACKGROUND OF THE DISCLOSURE

The purity of biologically produced therapeutics is tightly scrutinized and regulated by authorities to ensure safety and efficacy. Thus, there is a need to efficiently purify biologically produced therapeutics to a high degree of purity.

To support the clinical efforts for advanced therapy medicinal products (ATMPs), compositions and methods to efficiently purify ATMPs from recombinant sources are needed. Affinity purification is a means to isolate and/or achieve desired purity of a protein in a few steps, or in a single step. However, the development of separation matrices comprising an affinity ligand bound to a solid support can be a resource intensive and time-consuming task and hence affinity separation matrices exist for very few proteins. In the absence of an affinity separation matrix, purification typically involves inefficient processes, such as a multi-column process.

Adeno-associated virus (AAV), a member of the Parvovirus family, is a small, non-enveloped virus. AAV particles comprise an AAV capsid composed of 60 capsid protein subunits, VP1, VP2 and VP3, which enclose a single-stranded DNA genome of about 4.7 kilobases (kb). These VP1, VP2 and VP3 proteins are present in a predicted ratio of about 1:1:10 and are arranged in an icosahedral symmetry. Individual particles package only one DNA molecule strand, but this may be either the plus or minus strand, both of which are infectious. Unlike most viruses, AAVs are innately nonpathogenic, poorly immunogenic, and broadly tropic. Numerous AAV serotypes have been identified with variable tropisms. The tissue specificity of AAV is determined by the viral capsid serotype. This specificity allows the targeting of a gene of interest to certain tissues and cells. The properties of non-pathogenicity, a broad host range of infectivity, including non-dividing cells, and integration make the AAV serotypes, such as AAV8 an attractive delivery vehicle.

Recombinant adeno-associated viruses (rAAV) are one of the most investigated viral vectors for the delivery of gene therapies in humans. Recombinant AAV lacks two essential genes for viral integration and replication. As a result, rAAV remains primarily episomal and can persist in non-dividing cells for long periods of time. Because of these characteristics, along with the ability to target specific tissue types, recombinant AAV has become one of the main viral vectors used for research and gene therapy applications. AAV serotypes exhibit various cellular tropisms and interactions with cell receptors to allow entry into the cells and delivery of genetic cargo into the nucleus for expression. The manufacturing of rAAV is difficult and expensive. Cell culture productivity is low and typically only achieves 10¹³-10¹⁵ viral capsids per liter, which is equivalent to approximately 0.1-10 mg/L. Purification is mainly accomplished through the use of affinity chromatography. Currently, there are only four affinity resins available for the purification of AAV, POROS™ CaptureSelect™ AAV9, POROS™ CaptureSelect™ AAVX, POROS™ CaptureSelect™ AAV8, and AVB Sepharose. These resins have two major shortcomings in that they cannot be cleaned with sodium hydroxide and can only be reused for a few cycles. This increases resin consumption and leads to high resin costs for purification applications. Thus, there is a need for affinity agents with high specificity for AAV8 and AAV8 variants that are alkali stable and can support the production and purification of AAV8 and AAV8 variants.

### SUMMARY OF THE DISCLOSURE

Affinity ligands and affinity agents that bind AAV8 and are useful for isolation and/or affinity purification of AAV8 capsid and/or AAV8 variant capsid are described herein.

In one aspect, the disclosure provides an affinity ligand that specifically binds with AAV8 capsid or a variant of an AAV8 capsid, comprising the amino acid sequence represented by the formula, from N-terminus to C-terminus,

(SEQ ID NO: 1)
[A]-X₁QRRX₂FIX₃X₄LRX₅DPX₆X₇SX₈X₉LLX₁₀
X₁₁AX₁₂X₁₃X₁₄X₁₅X₁₆X₁₇-[B]

wherein
(a) [A] comprises a first α-helix-forming peptide domain;
(b) X₁ is A, R, N, S, D, L, Q or I, preferably R;
(c) X₂ is G, H, P or S, preferably S;
(d) X₃ is A or Y, preferably Y;
(e) X₄ is R or S, preferably R;
(f) X₅ is E, H or Q, preferably Q;
(g) X₆ is E or S, preferably S;
(h) X₇ is F, V or Y, preferably F;
(i) X₈ is A, E or R, preferably A;
(j) X₉ is H, I or N, preferably H;
(k) X₁₀ is A, E or R, preferably A;
(l) X₁₁ is D or E, preferably D;
(m) X₁₂ is K or R, preferably K;
(n) X₁₃ is Q, T or Y, preferably Y;
(o) X₁₄ is D, L or R, preferably R;
(p) X₁₅ is A or N, preferably N;
(q) X₁₆ is D, L or R, preferably R;
(r) X₁₇ is A, D, E, F, G, I, K, L, P, Q, R, S, T or Y, preferably I;
(s) [B] comprises a peptide comprising an amino acid sequence of QAPX₁₈ (SEQ ID NO: 2) or QAPX₁₈VD (SEQ ID NO: 3), wherein X₁₈ is A, K or R.

In certain embodiments, [A] comprises a peptide having an amino acid sequence of VDAKFDKELEEARAEIERLPNLTE (SEQ ID NO: 4) or VDAKFDKELEEIRAEIERLPNLTE (SEQ ID NO: 5). In certain embodiments, the N-terminus of [A] in the formula above is methionine (M) or MAQGT (SEQ ID NO: 6). In certain embodiments, [B] of the formula above has an amino acid sequence of QAPKVD (SEQ ID NO: 7) or QAPRVD (SEQ ID NO: 8). In other embodiments, [B] comprises a peptide having an amino acid sequence of QAPX₁₈-[C] (SEQ ID NO: 9), X₁₈ is A, K, or R and wherein [C] is a peptide domain selected from the group consisting of

(SEQ ID NO: 10)

| | |
|---|---|
| (a) | VDGQAGQGGGSGLNDIFEAQKIEWHEHHHHHH, |

(SEQ ID NO:11)

| | |
|---|---|
| (b) | GQAGQGGGSGLNDIFESEQ ID NO: 2AQKIEWHEHHHHHH, |

(SEQ ID NO: 12)

| | |
|---|---|
| (c) | VDGLNDIFEAQKIEWHEHHHHHH, |

and
(SEQ ID NO: 13)

| | |
|---|---|
| (d) | GLNDIFEAQKIEWHEHHHHHH. |

In certain embodiments, the affinity ligand further comprises a C-terminal lysine or cysteine.

In some embodiments, the affinity ligand comprises the sequence of any one of SEQ ID NOs:14-93. In other embodiments, the affinity ligand has the sequence of SEQ ID NO: 14 or SEQ ID NO: 53. In some embodiments, the affinity ligand has the amino acid sequence of SEQ ID NO: 54 and in other embodiments, the affinity ligand has the amino acid sequence of SEQ ID NO: 93.

In certain embodiments, the affinity ligand comprises at least one heterologous moiety operably linked to said affinity ligand to thereby form a conjugate. In certain embodiments, the heterologous moiety is one or more small molecule diagnostic or therapeutic agent; a DNA, RNA, or hybrid DNA-RNA molecule; a traceable marker; radioactive agent; an antibody; a single chain variable domain; or an immunoglobulin fragment.

In another aspect, there is provided a multimer comprising a plurality of affinity ligands according to any one of the aspects and embodiments herein. The multimer may be a dimer, trimer, tetramer, pentamer, hexamer, heptamer, octamer or nonamer.

In another aspect, there is provided a nucleic acid or vector encoding an affinity ligand or a multimer of any of the aspects and embodiments herein.

In another aspect, there is provided an expression system, comprising any of the nucleic acids or vectors of the disclosure.

In yet another aspect, there is provided a separation matrix, comprising at least one affinity ligand or at least one multimer of any of the aspects and embodiments herein. In certain embodiments, the separation matrix comprises a plurality of affinity ligands or multimers of any of the aspects and embodiments herein coupled to a solid support. In some embodiments, the affinity ligands or multimers of the affinity matrix are coupled to the solid support via carbamate bonds. In certain embodiments of this aspect, the solid support is a chromatographic resin or matrix. In certain embodiments, the solid support is a cross-linked agarose matrix.

In another aspect, there is provided a method of isolating adeno-associated virus subtype 8 (AAV8) particles or capsids, comprising contacting AAV8 particles or capsids with a separation matrix of the disclosure. In certain embodiments, the method comprises the steps of (a) contacting a liquid sample comprising AAV8 particles or capsids with the separation matrix, (b) washing said separation matrix with a washing liquid, (c) eluting the AAV8 particles or capsids from the separation matrix with an elution liquid, and (d) cleaning the separation matrix with a cleaning liquid. In certain embodiments of this aspect, the cleaning liquid comprises 0.1-0.5 M NaOH. In some embodiments of this aspect, steps (a)-(d) are repeated at least ten times.

### BRIEF DESCRIPTION OF THE DRAWINGS

FIG. 1 shows an example sensorgram for an exemplary affinity agent.

FIG. 2 shows exemplary stability data for certain affinity agents of the disclosure in the presence of 0.5 M NaOH

FIG. 3 shows an SD S-PAGE gel of viral particles purified using certain provided affinity agents. The elution (E) and strip (S) fractions were loaded on the gel. A reference standard (std) preparation of AAV capsids is included. The results for a ligand of the disclosure having an amino acid sequence of SEQ ID NO: 53 are shown.

FIG. 4 shows the effect of residence time on the binding capacity of a separation matrix containing affinity ligand of SEQ ID NO: 53 to bind to AAV8 capsids.

### DETAILED DESCRIPTION OF THE DISCLOSURE

### Definitions

In order for the present disclosure to be more readily understood, certain terms are defined below. Unless defined otherwise herein, technical and scientific terms used herein have the same meaning as commonly understood by one of ordinary skill in the art to which this disclosure is related.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

It is to be noted that the term “a” or “an” entity refers to one or more of that entity; for example, “an affinity ligand” is understood to represent one or more affinity ligands. As such, the terms “a” (or “an”), “one or more,” and “at least one” can be used interchangeably herein.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological or physiological effect on that organism, is considered to be biologically active.

Variant and Mutant: The term “variant” is usually defined in the scientific literature and used herein in reference to an organism that differs genetically in some way from an accepted standard, “Variant” can also be used to describe phenotypic differences that are not genetic (King and Stansfield, 2002, A dictionary of genetics, 6th ed., New York, New York, Oxford University Press.

The term “mutation” is defined by most dictionaries and used herein in reference to the process that introduces a heritable change into the structure of a gene (King & Stansfield, 2002) thereby producing a “mutant.” The term “variant” is increasingly being used in place of the term “mutation” in the scientific and non-scientific literature. The terms are used interchangeably herein.

Conservative and non-conservative substitution: A “conservative” amino acid substitution is one in which one amino acid residue is replaced with another amino acid residue having a similar side chain. Families of amino acid residues having similar side chains have been defined in the art, including basic side chains (e.g., lysine (K), arginine (R), histidine (H)); acidic side chains (e.g., aspartic acid (D), glutamic acid (E)); uncharged polar side chains (e.g., glycine (G); asparagine (N), glutamine (Q), serine (S), threonine (T), tyrosine (Y), cysteine (C)); nonpolar side chains (e.g., alanine (A), valine (V), leucine (L), isoleucine (I), proline (P), phenylalanine (F), menine (M), tryptophan (W), beta-branched side chains (e.g., threonine (T), valine (V), isoleucine (I)); and aromatic side chains (e.g., tyrosine (Y), phenylalanine (F), tryptophan (W), histidine (H)). For example, substitution of a phenylalanine for a tyrosine is a conservative substitution. In some embodiments, conservative amino acid substitutions in the sequence of a ligand confer or improve specific binding of the ligand a target of interest. In some embodiments, conservative amino acid substitutions in the sequences of a ligand do not reduce or abrogate the binding of the ligand to a target of interest. In some embodiments, conservative amino acid substitutions do not significantly affect specific binding of a ligand to a target of interest. Methods of identifying nucleotide and amino acid conservative substitutions and non-conservative substitutions which confer, alter or maintain selective binding affinity are known in the art (see, e.g., Brummell, Biochem. 32:1180-1187 (1993); Kobayashi, Protein Eng. 12(10):879-884 (1999); and Burks, PNAS 94:412-417 (1997)). In some embodiments, non-conservative amino acid substitutions in the sequence of a ligand confer or improve specific binding of the ligand a target of interest. In some embodiments, non-conservative amino acid substitutions in the sequences of a ligand do not reduce or abrogate the binding of the ligand to a target of interest. In some embodiments, non-conservative amino acid substitutions do not significantly affect specific binding of a ligand to a target of interest.

Affinity chromatography: As used herein the term “affinity chromatography” refers to the specific mode of chromatography in which an affinity ligand interacts with a target via biological affinity in a “lock-key” fashion. Examples of useful interactions in affinity chromatography are e.g., enzyme-substrate interaction, biotin-avidin interaction, antibody-antigen interaction, etc.

Affinity ligand and Ligand: The terms “affinity ligand” and “ligand” are used interchangeably herein. These terms are used herein to refer to molecules that are capable of reversibly binding with high affinity to a moiety specific for it, e.g., a polypeptide or protein.

Protein-based ligand: The term “protein-based ligands” as used herein means ligands which comprise a peptide or protein or a part of a peptide or protein that binds reversibly to a target polypeptide or protein. It is understood that the “ligands” of the disclosure are protein-based ligands.

Affinity agent: As used herein, the term “affinity agent” is in reference to a solid support or matrix to which a biospecific affinity ligand is covalently attached. Typically, the solid support or matrix is insoluble in the system in which the target molecule is purified. The terms “affinity agent” and “affinity separation matrix(ces)” and “separation matrix(ces)” are used interchangeably herein.

Linker: As used herein a “linker” refers to a peptide or other chemical linkage that functions to link otherwise independent functional domains. In some embodiments, a linker is located between a ligand and another polypeptide component containing an otherwise independent functional or structural domain. In some embodiments, a linker is a peptide or other chemical linkage located between a ligand and a surface.

Naturally occurring: The term “naturally occurring” when used in connection with biological materials such as a nucleic acid molecules, polypeptides, and host cells, refers to those which are found in nature and not modified by a human being. Conversely, “non-natural” or “synthetic” when used in connection with biological materials refers to those which are not found in nature and/or have been modified by a human being.

“Non-natural amino acids,” “amino acid analogs” and “non-standard amino acid residues” are used interchangeably herein. Non-natural amino acids that can be substituted in a ligand as provided herein are known in the art. In some embodiments, a non-natural amino acid is 4-hydroxyproline which can be substituted for proline; 5-hydroxylysine which can be substituted for lysine; 3-methylhistidine which can be substituted for histidine; homoserine which can be substituted for serine; and ornithine which can be substituted for lysine. Additional examples of non-natural amino acids that can be substituted in a polypeptide ligand include, but are not limited to molecules such as: D-isomers of the common amino acids, 2,4-diaminobutyric acid, alpha-amino isobutyric acid, A-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, lanthionine, dehydroalanine, γ-aminobutyric acid, selenocysteine and pyrrolysine fluoro-amino acids, designer amino acids such as beta-methyl amino acids, C alpha-methyl amino acids, and N alpha-methyl amino acids.

“Polynucleotide” and “nucleic acid molecule”: As used interchangeably herein, polynucleotide and nucleic acid molecule refer to a polymeric form of nucleotides of any length, either ribonucleotides or deoxyribonucleotides. These terms include, but are not limited to, DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA (transfer RNA).

Operably linked: The term “operably linked,” as used herein, indicates that two or more components are arranged such that the components function normally and allow the possibility that at least one of the components can mediate a function that is exerted upon at least one of the other components. Two molecules are “operably linked” whether they are attached directly or indirectly.

Peptide tag: The term “peptide tag” as used herein refers to a peptide sequence that is part of or attached (for instance through genetic engineering) to another protein, to provide a function to the resultant fusion. Peptide tags are usually relatively short in comparison to a protein to which they are fused. In some embodiments, a peptide tag is four or more amino acids in length, such as, 5, 6, 7, 8, 9, 10, 15, 20, or 25 or more amino acids. In some embodiments, a ligand is a protein that contains a peptide tag. Numerous peptide tags that have uses as provided herein are known in the art. Examples of peptide tags that may be a component of a ligand fusion protein or a target bound by a ligand (e.g., a ligand fusion protein) include but are not limited to HA (hemagglutinin), c-myc, the Herpes Simplex virus glycoprotein D (gD), T7, GST, GFP, MBP, Strep-tags, His-tags, Myc-tags, TAP-tags and FLAG tag (Eastman Kodak, Rochester, N.Y.) Likewise, antibodies to the tag epitope allow detection and localization of the fusion protein in, for example, affinity purification, Western blots, ELISA assays, and immunostaining of cells.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Specifically binds: As used herein in reference to ligands, the term “specifically binds” or “has selective affinity for” means a ligand reacts or associates more frequently, more rapidly, with greater duration, with greater affinity, or combinations of the above to a particular epitope, protein, or target molecule than with alternative substances, including unrelated proteins. Because of the sequence identity between homologous proteins in different species, specific binding can include a binding agent that recognizes a protein or target in more than one species, e.g., is bi- or tri-specific. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, i.e., binding to a single target. Thus, a ligand or affinity agent may, in certain embodiments, specifically bind more than one target. In certain embodiments, multiple targets may be bound by the same binding site on an affinity agent.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The present disclosure encompasses, inter alia, the use of affinity agents comprising peptide ligands attached to a solid support to generate highly purified preparations of one or more targets of interest, such as for example, adeno-associated virus (AAV) particles, more particularly AAV8 particles. In some embodiments, affinity agents described herein are useful for, inter alia, removal of protein product related impurities as well as host cell derived contaminants.

### AAV8 Affinity Ligands

The ligands of the various aspects and embodiments of the disclosure are high affinity protein ligands that reversibly bind to AAV8 capsid and/or AAV8 variant capsid. A number of AAV8 variants are known in the art. (e.g., AAV8 variant (Y733F, Y447F, Y447F) GeneMedi; See also Gilkes, et al., Site-specific modifications to AAV8 capsid yields enhanced brain transduction in the neonatal MPS IIIB mouse, Gene therapy, 28: 447-455 (2021). The targeted AAV8 may be a naturally occurring or recombinant virus particle. Non-limiting uses of the targeted molecule include therapeutic and diagnostic uses.

The affinity ligands of the disclosure have the general formula:

(SEQ ID NO: 1)
[A]-X₁QRRX₂FIX₃X₄LRX₅DPX₆X₇SX₈X₉LLX₁₀
X₁₁AX12X₁₃X₁₄X₁₅X₁₆X₁₇-[B],

wherein
[A] comprises a first α-helix-forming peptide domain; X₁ is A, R, N, S, D, L, Q or I, preferably R;

X₂ is G, H, P or S, preferably S; X₃ is A or Y, preferably Y; X₄ is R or S, preferably R; X₅ is E, H or Q, preferably Q; X₆ is E or S, preferably S; X₇ is F, V or Y, preferably F; X₅ is A, E or R, preferably A; X₉ is H, I or N, preferably H; X₁₀ is A, E or R, preferably A; X₁₁ is D or E, preferably D; X₁₂ is K or R, preferably K; X₁₃ is Q, T or Y, preferably Y; X₁₄ is D, L or R, preferably R; X₁₅ is A or N, preferably N; X₁₆ is D, L or R, preferably R; X₁₇ is A, D, E, F, G, I, K, L, P, Q, R, S, T or Y, preferably I; and [B] is comprises a peptide comprising an amino acid sequence of QAPX₁₈ (SEQ ID NO: 2) or QAPX₁₈VD (SEQ ID NO: 3), wherein X₁₈ is A, K or R.

Moiety [A] of the formula above is a peptide that provides an α-helical structure to the N-terminal end of the ligand. In some embodiments, [A] has the amino acid sequence of VDAKFDKELEEARAEIERLPNLTE (SEQ ID NO: 4). In other embodiments, [A] has the amino acid sequence of VDAKFDKELEEIRAEIERLPNLTE (SEQ ID NO: 5). The N-terminus of the affinity ligand (i.e., the N-terminus of [A]) may be a methionine or may include an additional amino acid sequence of MAQGT (SEQ ID NO: 6).

Moiety [B] of the formula for the affinity ligands of the disclosure may have the amino acid sequence of QAPKVD (SEQ ID NO: 7) or QAPRVD (SEQ ID NO: 8), for example. In other embodiments, [B] has the amino acid sequence QAPXis-[C] (SEQ ID NO: 9), wherein [C] is a peptide having the amino acid sequence of VDGQAGQGGGSGLNDIFEAQKIEWHEHEIREIHH, (SEQ ID NO: 10); GQAGQ GGGS GLNDIFEAQKIEWHEHREIRHE (SEQ ID NO: 11); VDGLNDIFEAQKIEWHEHHHHHH (SEQ ID NO: 12) or GLNDIFEAQKIEWHEHREIHHH (SEQ ID NO: 13) and X₁₈ is A, K, or R.

The affinity ligand may have the amino acid sequence of any one of SEQ ID Nos: 14-93. In certain embodiments, the affinity ligand has the amino acid sequence of SEQ ID NO: 14, while in other embodiments, the affinity ligand has the amino acids sequence of SEQ ID NO: 53. In some embodiments, the affinity ligand has the amino acid sequence of SEQ ID NO: 54. In some embodiments, the affinity ligand has the amino acid sequence of SEQ ID NO: 93.

In another aspect, the present disclosure provides a multimer comprising, a plurality of affinity ligands (units) as defined by any embodiment disclosed above. The multimer may be a dimer, a trimer, a tetramer, a pentamer, a hexamer, a heptamer, an octamer or a nonamer. The multimer may be a homomultimer where all of the ligand units are identical or the multimer may be a heteromultimer, where at least one ligand unit differs from the others. The ligands may be linked to each other directly by peptide bonds between the C-terminal and N-terminal ends of the ligand. Alternatively, two or more ligand units of the multimer may be linked by linkers comprising oligomeric or polymeric species, such as elements comprising up to 15 or 30 amino acids, such as 1-5, 1-10 or 5-10 amino acids.

In some embodiments of this aspect, the multimers of the disclosure have the general structural formula:

(SEQ ID NO: 95)
[[A]- core-QAPX]n-[C],

where [A] is VDAKFDKELEEARAEIERLPNLTE (SEQ ID NO: 4), [C] is VDGLNDIFEAQKIEWHEHREIRREI (SEQ ID NO: 12), and core is X₁QRRX₂FIX₃X₄LRX₅DPX₆X₇SX₈X₉LLX₁₀X₁₁AX₁₂X₁₃X₁₄X₁₅X₁₆X₁₇, wherein X₁-X₁₇ are the same as defined above for the affinity ligands, X₁₅ A, K, or R and n is any number between 2 and 10. (SEQ ID NO: 96). Optionally, the N-terminus of the structure is M or MAQGT (SEQ ID NO: 6).

In some embodiments, the ligand and/or multimer as disclosed above, further comprises at the C-terminal or N-terminal end one or more coupling elements, such as one or more cysteine residues, one or more lysine residues or a plurality of histidine residues. In certain embodiments, the affinity ligand and/or multimer further comprises a heterologous agent operably linked to the ligand and/or multimer, such as for example one or more of a small molecule diagnostic or therapeutic; DNA, RNA, or hybrid DNA-RNA; traceable marker; radioactive agent; an antibody; a single chain variable domain; or an immunoglobulin fragment.

### Ligand Binding to AAV8

The characteristics of a ligand or multimer that binds to a target, such as AAV8 and/or AAV8 variants can be determined using known or modified assays, bioassays, and/or animal models known in the art for evaluating such activity.

As used herein, terms such as “binding affinity for a target,” “binding to a target, “binding to AAV8 or an AAV8 variant,” and the like refer to a property of a ligand of the disclosure which may be directly measured, for example, through the determination of affinity constants (e.g., the amount of ligand that associates and dissociates at a given antigen concentration). Several methods are available to characterize such molecular interactions, for example, competition analysis, equilibrium analysis and microcalorimetric analysis, and real-time interaction analysis based on surface plasmon resonance interaction (for example using a BIACORE instrument). These methods are well-known to those of skill in the art and are discussed in publications such as Neri D et al. (1996) Tibtech 14:465-470 and Jansson M et al. (1997) J Biol Chem 272:8189-8197.

Affinity requirements for a given ligand binding event are contingent on a variety of factors including, but not limited to the composition and complexity of the binding matrix, the valency and density of both the ligand and target molecules, and the functional application of the ligand. In some embodiments, a ligand of the disclosure binds AAV8 or a variant of AAV8 with a dissociation constant (KD) of less than or equal to 5×10⁻³ M, 10⁻³ M, 5×10⁻⁴ M, 10⁻⁴ M, 5×10⁻⁵ M, or 10⁻⁵ M. In some embodiments, a ligand binds a target of interest with a KD of less than or equal to 5×10⁻⁶ M, 10⁻⁶ M, 5×10⁻⁷ M, 10⁻⁷ M, 5×10⁻⁸ M, or 10⁻⁸ M. In some embodiments, a ligand binds a target of interest with a KD less than or equal to 5×10⁻⁹ M, 10⁻⁹ M, 5×10⁻¹⁰ M, 10−10 M, 5×10⁻¹¹ M, 10⁻¹¹ M 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In some embodiments, a ligand generated by methods disclosed herein has a dissociation constant of from about 10⁻⁴ M to about 10⁻⁵ M, from about 10⁻⁵ M to about 10⁻⁶ M, from about 10⁻⁶ M to about 10⁻⁷ M, from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M.

In some embodiments, a ligand or multimer of the disclosure specifically binds an AAV8 particle or capsid or an AAV8 variant particle or capsid with a koff ranging from 0.1 to 10⁻⁷ sec⁻¹, 10⁻² to 10⁻⁷ sec⁻¹, or 0.5×10⁻² to 10⁻³ sec⁻¹. In some embodiments, a ligand binds a target of interest with an off rate (koff) of less than 5×10⁻² sec⁻¹, 10⁻² sec⁻¹, 5×10⁻³ sec⁻¹, or 10⁻³ sec⁻¹. In some embodiments a ligand binds a target of interest with an off rate (koff) of less than 5×10⁻⁴ sec⁻¹, 10⁻⁴ sec⁻¹, 5×10″ 5 sec⁻¹, or 10⁻⁵ sec⁻¹, 5×10⁻⁶ sec⁻¹, 10″ 6 sec⁻¹, 5×10⁻⁷ sec⁻¹, or 10⁻⁷ sec⁻¹.

In some embodiments, a ligand or multimer specifically binds an AAV8 particle or capsid or an AAV8 variant particle or capsid with a kon ranging from about 10³ to 10⁷ M⁻¹ sec⁻¹, 10³ to 10⁶ M⁻¹ sec⁻¹, or 10³ to 10⁵ M⁻¹ sec⁻¹. In some embodiments, a ligand (e.g., a ligand fusion protein) binds the target of interest with an on rate (kon) of greater than 10³ M⁻¹ sec⁻¹, 5×10³ M⁻¹ sec⁻¹, 10⁴ M⁻¹ sec⁻¹, or 5×10⁴ M⁻¹ sec⁻¹. In an additional embodiment, a ligand, binds a target of interest with a kon of greater than 10⁵ M⁻¹ sec⁻¹, 5×10 5 M⁻¹ sec⁻¹, 10⁶ M⁻¹ sec⁻¹, 5×10⁶ M⁻¹ sec⁻¹, or 10⁷ M⁻¹ sec⁻¹.

### Linkers

The terms “linker” and “spacer” are used interchangeably herein to refer to a peptide or other chemical linkage that functions to link otherwise independent functional domains. In some embodiments, a linker is located between a ligand and another polypeptide component containing an otherwise independent functional domain. Suitable linkers for coupling two or more linked ligands may generally be any linker used in the art to link peptides, proteins or other organic molecules. In some embodiments, such a linker is suitable for constructing proteins or polypeptides that are intended for pharmaceutical use.

Suitable linkers for operably linking a ligand and an additional component of a ligand fusion protein in a single-chain amino acid sequence include but are not limited to, polypeptide linkers such as glycine linkers, serine linkers, mixed glycine/serine linkers, glycine- and serine-rich linkers or linkers composed of largely polar polypeptide fragments.

In some embodiments, a linker comprises a majority of amino acids selected from glycine, alanine, proline, asparagine, glutamine, and lysine. In some embodiments, a linker comprises a majority of amino acids selected from glycine, alanine, proline, asparagine, aspartic acid, threonine, glutamine, and lysine. In some embodiments, a ligand linker is made up of a majority of amino acids that are sterically unhindered. In some embodiments, a linker comprises a majority of amino acids selected from glycine, serine, and/or alanine. In some embodiments, a linker is selected from polyglycines (such as (Gly)5, and (Gly)8, poly(Gly-Ala), and polyalanines.

Linkers can be of any size or composition so long as they are able to operably link a ligand in a manner that permits the ligand to bind a target of interest. In some embodiments, linkers are from about 1 to 50 amino acids, from about 1 to 20 amino acids, from about 1 to 15 amino acids, from about 1 to 10 amino acids, from about 1 to 5 amino acids, from about 2 to 20 amino acids, from about 2 to 15 amino acids, from about 2 to 10 amino acids, or from about 2 to 5 amino acids. It should be clear that the length, the degree of flexibility and/or other properties of the linker(s) may influence certain properties of a ligand for use in an affinity agent, such as affinity, specificity or avidity for a target of interest, or for one or more other target proteins of interest, or for proteins not of interest (i.e., non-target proteins). In some embodiments, two or more linkers are utilized. In some embodiments, two or more linkers are the same. In some embodiments, two or more linkers are different.

In some embodiments, a linker is a non-peptide linker such as an alkyl linker, or a PEG linker. For example, alkyl linkers such as —NH—(CH₂)ₛ—C(O)—, wherein s=2-20 can be used. These alkyl linkers may further be substituted by any non-sterically hindering group such as lower alkyl e.g., C₁ C₆) lower acyl, halogen (e.g., CI, Br), CN, NH₂, phenyl, etc. An exemplary non-peptide linker is a PEG linker. In some embodiments, a PEG linker has a molecular weight of from about 100 to 5000 kDa, or from about 100 to 500 kDa.

Linkers can be evaluated using techniques described herein and/or otherwise known in the art. In some embodiments, linkers do not alter (e.g., do not disrupt) the ability of a ligand to bind a target molecule.

### Affinity Agents Comprising Conjugated Ligands: Affinity Separation Matrices

Ligands or multimers that promote specific binding to targets of interest can be chemically conjugated to a variety of surfaces used in chromatography, e.g., beads, resins, gels, membrane, monoliths, etc. to prepare an affinity agent. Affinity agents of the disclosure are particularly useful for AAV8 and AAV8 variant purification and manufacturing applications.

In some embodiments, a ligand of the disclosure (e.g., a ligand fusion protein) contains at least one reactive residue. Reactive residues are useful, for example, as sites for the attachment of conjugates such as chemotherapeutic drugs or diagnostic agents. Exemplary reactive amino acid residues include lysine or cysteine, for example. A reactive residue can be added to a ligand at either end, or within the ligand sequence and/or can be substituted for another amino acid within the ligand sequence. A suitable reactive residue (e.g., lysine, cysteine, etc.) can also be located within the sequence of an identified ligand without need for addition or substitution.

### Attachment to a Solid Surface

“Solid surface,” “support,” or “matrix” are used interchangeably herein and refer to, without limitation, any column (or column material), bead, test tube, microtiter dish, solid particle (for example, agarose or sepharose), microchip (for example, silicon, silicon-glass, or gold chip), or membrane (synthetic (e.g. a filter) or biological (e.g. liposome or vesicle) in origin to which a ligand or multimer of the disclosure may be attached (i.e., coupled, linked, or adhered), either directly or indirectly (for example, through other binding partner intermediates such as a linker), or in which a ligand or multimer may be embedded (for example, through a receptor or channel). Reagents and techniques for attaching polypeptides to solid supports are well-known in the art, e.g., carbamate coupling. Suitable solid supports include, but are not limited to, a chromatographic resin or matrix (e.g., SEPHAROSE-4 FF agarose beads), the wall or floor of a well in a plastic microtiter dish, a silica-based biochip, polyacrylamide, agarose, silica, nitrocellulose, paper, plastic, nylon, metal, and combinations thereof. Ligands and other compositions may be attached on a support material by a non-covalent association or by covalent bonding, using reagents and techniques known in the art. In some embodiments, a ligand is coupled to a chromatography material using a linker.

In one aspect, the disclosure provides an affinity agent (affinity separation matrix) comprised of a ligand or multimer as described above coupled to an insoluble support. Such a support may be one or more particles, such as beads; membranes; filters; capillaries; monoliths; and any other format commonly used in chromatography. In an advantageous embodiment of the affinity separation matrix, the support is comprised of substantially spherical particles, also known as beads. Suitable particle sizes may be in the diameter range of 5-500 μm, such as 10-100 μm, e.g., 20-80 μm. In an alternative embodiment, the support is a membrane. To obtain high adsorption capacities, the support is preferably porous, and ligands may be coupled to the external surfaces as well as to the pore surfaces. In an advantageous embodiment of this aspect, the support is porous.

In another aspect, the disclosure relates to a method of preparing a chromatography affinity agent, which method comprises providing ligands as described above, and coupling the ligands to a support. Coupling may be carried out via a nitrogen or sulfur atom of the ligand for example. The ligands may be coupled to the support directly or indirectly via a spacer element to provide an appropriate distance between the support surface and the ligand. Methods for immobilization of protein ligands to porous or non-porous surfaces are well known in this field.

### Production of Ligands

The production of ligands and multimers, useful in practicing several embodiments of the disclosure, may be carried out using a variety of standard techniques for chemical synthesis, semi-synthetic methods, and recombinant DNA methodologies known in the art. Also provided are methods for producing a ligand or multimer, individually or as part of multi-domain fusion protein, as soluble agents and cell associated proteins. In some embodiments, the overall production scheme for a ligand or multimer comprises obtaining a reference protein scaffold and identifying a plurality of residues within the scaffold for modification. Depending on the embodiment, the reference scaffold may comprise a protein structure with one or more alpha-helical regions, or other tertiary structure. Once identified, any of a plurality of residues can be modified, for example by substitution of one or more amino acids. In some embodiments, one or more conservative substitutions are made. In some embodiments, one or more non-conservative substitutions are made. In some embodiments a natural amino acid (e.g., one of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine) is substituted into a reference scaffold at targeted positions for modification. In some embodiments, modifications do not include substituting in either a cysteine or a proline. After modifications have been made at identified positions desired in a particular embodiment, the resulting modified polypeptides (e.g., candidate ligands) can be recombinantly expressed, for example in a plasmid, bacteria, phage, or other vector (e.g., to increase the number of each of the modified polypeptides). The modified polypeptides can then be purified and screened to identify those modified polypeptides that have specific binding to a particular target of interest, e.g., AAV8 or variant of AAV8. Modified polypeptides may show enhanced binding specificity for AAV8 or variant of AAV8 as compared to a reference scaffold, or may exhibit little or no binding to a given target of interest (or to a non-target protein). In some embodiments, depending on the target of interest, the reference scaffold may show some interaction (e.g., nonspecific interaction) with the target of interest, while certain modified polypeptides will exhibit at least about two-fold, at least about five-fold, at least about tenfold, at least about 20-fold, at least about 50-fold, or at least about 100-fold (or more) increased binding specificity for the target of interest. Additional details regarding production, selection, and isolation of ligand are provided in more detail below.

### Recombinant Expression of Ligands

In some embodiments, a ligand such as a ligand fusion protein is “recombinantly produced,” (i.e., produced using recombinant DNA technology). Exemplary recombinant methods available for synthesizing ligand fusion proteins, include, but are not limited to polymerase chain reaction (PCR) based synthesis, concatemerization, seamless cloning, and recursive directional ligation (RDL) (see, e.g., Meyer et al., Biomacromolecules 3:357-367 (2002), Kurihara et al., Biotechnol. Lett. 27:665-670 (2005), Haider et al., Mol. Pharm. 2:139-150 (2005); and McMillan et al., Macromolecules 32(11):3643-3646 (1999).

In another aspect, nucleic acids comprising a polynucleotide sequence encoding a ligand or multimer according to the embodiments disclosed above are also provided. Thus, the disclosure encompasses all forms of the present nucleic acid sequence such as RNA and DNA encoding the polypeptide (ligand) or multimer. The disclosure provides vectors, such as plasmids, which in addition to the coding sequence comprise the required signal sequences for expression of the polypeptide or multimer according the disclosure. Such polynucleotides optionally further comprise one or more expression control elements. For example, a polynucleotide can comprise one or more promoters or transcriptional enhancers, ribosomal binding sites, transcription termination signals, and polyadenylation signals, as expression control elements. A polynucleotide can be inserted within any suitable vector, which can be contained within any suitable host cell for expression. In one embodiment, the vector comprises nucleic acid encoding a multimer according to the disclosure, wherein the separate nucleic acids encoding each unit may have homologous or heterologous DNA sequences.

The expression of nucleic acids encoding ligands and multimers is typically achieved by operably linking a nucleic acid encoding the ligand to a promoter in an expression vector. Typical expression vectors contain transcription and translation terminators, initiation sequences, and promoters useful for regulation of the expression of the desired nucleic acid sequence. Exemplary promoters useful for expression in E. coli include, for example, the T7 promoter.

Methods known in the art can be used to construct expression vectors containing the nucleic acid sequence encoding a ligand along with appropriate transcriptional/translational control signals. These methods include, but are not limited to in vitro recombinant DNA techniques, synthetic techniques and in vivo recombination/genetic recombination. The expression of the polynucleotide can be performed in any suitable expression host known in the art including, but not limited to, bacterial cells, yeast cells, insect cells, plant cells or mammalian cells. In some embodiments, a nucleic acid sequence encoding a ligand is operably linked to a suitable promoter sequence such that the nucleic acid sequence is transcribed and/or translated into ligand in a host.

A variety of host-expression vector systems can be utilized to express a nucleic acid encoding a ligand. Vectors containing the nucleic acids encoding a ligand (e.g., individual ligand subunits or ligand fusions) or portions or fragments thereof, include plasmid vectors, a single and double-stranded phage vectors, as well as single and double-stranded RNA or DNA viral vectors. Phage and viral vectors may also be introduced into host cells in the form of packaged or encapsulated virus using known techniques for infection and transduction. Moreover, viral vectors may be replication competent or alternatively, replication defective. Alternatively, cell-free translation systems may also be used to produce the protein using RNAs derived from the DNA expression constructs (see, e.g., WO86/05807 and WO89/01036; and U.S. Pat. No. 5,122,464).

Generally, any type of cell or cultured cell line can be used to express a ligand or multimer provided herein. In some embodiments a background cell line used to generate an engineered host cell is a phage, a bacterial cell, a yeast cell or a mammalian cell. A variety of host-expression vector systems may be used to express the coding sequence a ligand fusion protein. Mammalian cells can be used as host cell systems transfected with recombinant plasmid DNA or cosmid DNA expression vectors containing the coding sequence of the target of interest and the coding sequence of the fusion polypeptide. The cells can be primary isolates from organisms, cultures, or cell lines of transformed or transgenic nature.

Suitable host cells include but are not limited to microorganisms such as, bacteria (e.g., E. coli, B. subtilis) transformed with recombinant bacteriophage DNA, plasmid DNA or cosmid DNA expression vectors containing ligand coding sequences; yeast (e.g., Saccharomyces, Pichia) transformed with recombinant yeast expression vectors containing ligand coding sequences; insect cell systems infected with recombinant virus expression vectors (e.g., Baculovirus) containing ligand coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing ligand coding sequences.

Prokaryotes useful as host cells in producing a ligand include gram negative or gram positive organisms such as, E. coli and B. subtilis. Expression vectors for use in prokaryotic host cells generally contain one or more phenotypic selectable marker genes (e.g., genes encoding proteins that confer antibiotic resistance or that supply an autotrophic requirement). Examples of useful prokaryotic host expression vectors include the pKK223-3 (Pharmacia, Uppsala, Sweden), pGEM1 (Promega, Wis., USA), pET (Novagen, Wis., USA) and pRSET (Invitrogen, Calif, USA) series of vectors (see, e.g., Studier, J. Mol. Biol. 219:37 (1991) and Schoepfer, Gene 124:83 (1993)). Exemplary promoter sequences frequently used in prokaryotic host cell expression vectors include T7, (Rosenberg et al., Gene 56:125-135 (1987)), beta-lactamase (penicillinase), lactose promoter system (Chang et al., Nature 275:615 (1978)); and Goeddel et al., Nature 281:544 (1979)), tryptophan (trp) promoter system (Goeddel et al., Nucl. Acids Res. 8:4057, (1980)), and tac promoter (Sambrook et al., 1990, Molecular Cloning, A Laboratory Manual, 2d Ed., Cold Spring Harbor Laboratory, Cold Spring Harbor, N.Y.).

In some embodiments, a eukaryotic host cell system is used, including yeast cells transformed with recombinant yeast expression vectors containing the coding sequence of a ligand. Exemplary yeast that can be used to produce compositions of the disclosure, include yeast from the genus Saccharomyces, Pichia, Actinomycetes and Kluyveromyces. Yeast vectors typically contain an origin of replication sequence from a 2 mu yeast plasmid, an autonomously replicating sequence (ARS), a promoter region, sequences for polyadenylation, sequences for transcription termination, and a selectable marker gene. Examples of promoter sequences in yeast expression constructs include promoters from metallothionein, 3-phosphoglycerate kinase (Hitzeman, J. Biol. Chem. 255:2073 (1980)) and other glycolytic enzymes, such as, enolase, glyceraldehyde-3-phosphate dehydrogenase, hexokinase, pyruvate decarboxylase, phosphofructokinase, glucose-6-phosphate isomerase, 3-phospho glycerate mutase, pyruvate kinase, triosephosphate isomerase, phosphoglucose isomerase, and glucokinase. Additional suitable vectors and promoters for use in yeast expression as well as yeast transformation protocols are known in the art. See, e.g., Fleer, Gene 107:285-195 (1991) and Hinnen, PNAS 75:1929 (1978).

Insect and plant host cell culture systems are also useful for producing the compositions of the disclosure. Such host cell systems include for example, insect cell systems infected with recombinant virus expression vectors (e.g., baculovirus) containing the coding sequence of a ligand; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of a ligand, including, but not limited to, the expression systems taught in U.S. Pat. No. 6,815,184; U.S. Publ. Nos. 60/365,769, and 60/368,047; and WO2004/057002, WO2004/024927, and WO2003/078614.

In some embodiments, host cell systems may be used, including animal cell systems infected with recombinant virus expression vectors (e.g., adenoviruses, retroviruses, adeno-associated viruses, herpes viruses, lentiviruses) including cell lines engineered to contain multiple copies of the DNA encoding a ligand either stably amplified (CHO/dhfr) or unstably amplified in double-minute chromosomes (e.g., murine cell lines). In some embodiments, a vector comprising a polynucleotide(s) encoding a ligand is polycistronic. Exemplary mammalian cells useful for producing these compositions include 293 cells (e.g., 293T and 293F), CHO cells, BHK cells, NSO cells, SP2/0 cells, YO myeloma cells, P3X63 mouse myeloma cells, PER cells, PER.C6 (Crucell, Netherlands) cells VERY, Hela cells, COS cells, MDCK cells, 3T3 cells, W138 cells, BT483 cells, Hs578T cells, HTB2 cells, BT20 cells, T47D cells, CRL7030 cells, HsS78Bst cells, hybridoma cells, and other mammalian cells. Additional exemplary mammalian host cells that are useful in practicing the embodiments of the disclosure include but are not limited, to T cells. Exemplary expression systems and selection methods are known in the art and, including those described in the following references and references cited therein: Borth et al., Biotechnol. Bioen. 71(4):266-73 (2000), in Werner et al., Arzneimittelforschung/Drug Res. 48(8):870-80 (1998), Andersen et al., Curr. Op. Biotechnol. 13:117-123 (2002), Chadd et al., Curr. Op, Biotechnol. 12:188-194 (2001), and Giddings, Curr. Op. Biotechnol. 12:450-454 (2001). Additional examples of expression systems and selection methods are described in Logan et al., PNAS 81:355-359 (1984), Birtner et al. Methods Enzymol. 153:51-544 (1987)). Transcriptional and translational control sequences for mammalian host cell expression vectors are frequently derived from viral genomes. Commonly used promoter sequences and enhancer sequences in mammalian expression vectors include, sequences derived from Polyoma virus, Adenovirus 2, Simian Virus 40 (SV40), and human cytomegalovirus (CMV). Exemplary commercially available expression vectors for use in mammalian host cells include pCEP4 (Invitrogen) and pcDNA3 (Invitrogen).

Physical methods for introducing a nucleic acid into a host cell (e.g., a mammalian host cell) include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian (e.g., human) cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Methods for introducing a DNA and RNA polynucleotides of interest into a host cell include electroporation of cells, in which an electrical field is applied to cells in order to increase the permeability of the cell membrane, allowing chemicals, drugs, or polynucleotides to be introduced into the cell. Ligand containing DNA or RNA constructs may be introduced into mammalian or prokaryotic cells using electroporation.

In some embodiments, electroporation of cells results in the expression of a ligand-CAR on the surface of T cells, NK cells, NKT cells. Such expression may be transient or stable over the life of the cell. Electroporation may be accomplished with methods known in the art including MaxCyte GT® and STX® Transfection Systems (MaxCyte, Gaithersburg, MD, USA).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid is associated with a lipid. A nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristoyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristoyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-510 (1991)). However, compositions that have different structures in solution than the normal vesicular structure are also encompassed. For example, the lipids can assume a micellar structure or merely exist as non-uniform aggregates of lipid molecules. Also contemplated are lipofectamine-nucleic acid complexes.

Regardless of the method used to introduce exogenous nucleic acids into a host cell, the presence of the recombinant nucleic acid sequence in the host cell can routinely be confirmed through a variety of assays known in the art. Such assays include, for example, “molecular biological” assays, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Reporter genes are used for identifying potentially transfected cells and for evaluating the functionality of regulatory sequences. In general, a reporter gene is a gene that is not present in or expressed by the recipient organism, tissue, or cell and that encodes a polypeptide whose expression is manifested by some easily detectable property, e.g., enzymatic activity. Expression of the reporter gene is assayed at a suitable time after the DNA has been introduced into the recipient cells. Suitable reporter genes include, but are not limited to, genes encoding luciferase, beta-galactosidase, chloramphenicol acetyl transferase, secreted alkaline phosphatase, or the green fluorescent protein gene (e.g., Ui-Tei et al., FEBS Lett. 479:79-82 (2000)). Suitable expression systems are known in the art and can be prepared using known techniques or obtained commercially. In general, the construct with the minimal 5′ flanking region showing the highest level of expression of reporter gene is identified as the promoter. Such promoter regions can routinely be linked to a reporter gene and used to evaluate agents for the ability to modulate promoter-driven transcription.

A number of selection systems can be used in mammalian host-vector expression systems, including, but not limited to, the herpes simplex virus thymidine kinase, hypoxanthine-guanine phosphoribosyltransferase and adenine phosphoribosyltransferase (Lowy et al., Cell 22:817 (1980)) genes. Additionally, antimetabolite resistance can be used as the basis of selection for e.g., dhfr, gpt, neo, hygro, trpB, hisD, ODC (ornithine decarboxylase), and the glutamine synthase system.

In some embodiments, the initiator N-terminal methionine is included at the NH-terminus of the ligand. In many instances the ligand is isolated without the N-terminal methionine residue, which is presumed to be cleaved during expression. In many instances a mixture is obtained with only a proportion of the purified ligand contains the N-terminal methionine. It is obvious to those skilled in the art that the presence or absence of the N-terminal methionine does not affect the functionality of the ligands and affinity agents described herein.

### Ligand Purification

Once a ligand or a ligand fusion protein or multimer has been produced by recombinant expression, it can be purified by methods known in the art for purification of a recombinant protein, for example, by chromatography (e.g., ion exchange, affinity, and sizing column chromatography), centrifugation, differential solubility, or by any other standard technique for the purification of proteins. In some embodiments, a ligand is optionally fused to heterologous polypeptide sequences specifically disclosed herein or otherwise known in the art to facilitate purification. In some embodiments, ligands (e.g., antibodies and other affinity matrices) for ligand affinity columns for affinity purification and that optionally, the ligand or other components of the ligand fusion composition that are bound by these ligands are removed from the composition prior to final preparation of the ligand using techniques known in the art.

### Chemical Synthesis of Ligand

In addition to recombinant methods, ligand production may also be carried out using organic chemical synthesis of the desired polypeptide using a variety of liquid and solid phase chemical processes known in the art. Various automatic synthesizers are commercially available and can be used in accordance with known protocols. See, for example, Tam et al., J. Am. Chem. Soc., 105:6442 (1983); Merrifield, Science, 232:341-347 (1986); Barany and Merrifield, The Peptides, Gross and Meienhofer, eds, Academic Press, New York, 1-284; Barany et al., Int. J. Pep. Protein Res., 30:705 739 (1987); Kelley et al. in Genetic Engineering Principles and Methods, Setlow, J. K., ed. Plenum Press, N Y. 1990, vol. 12, pp. 1-19; Stewart et al., Solid-Phase Peptide Synthesis, W.H. Freeman Co., San Francisco, 1989. One advantage of these methodologies is that they allow for the incorporation of non-natural amino acid residues into the sequence of the ligand.

The ligands and multimers that are used in the methods of the disclosure may be modified during or after synthesis or translation, e.g., by glycosylation, acetylation, benzylation, phosphorylation, amidation, pegylation, formylation, derivatization by known protecting/blocking groups, proteolytic cleavage, linkage to an antibody molecule, hydroxylation, iodination, methylation, myristoylation, oxidation, pegylation, proteolytic processing, phosphorylation, prenylation, racemization, selenoylation, sulfation, ubiquitination, etc. (See, e.g., Creighton, Proteins: Structures and Molecular Properties, 2d Ed. (W.H. Freeman and Co., N.Y., 1992); Postranslational Covalent Modification of Proteins, Johnson, ed. (Academic Press, New York, 1983), pp. 1-12; Seifter, Meth. Enzymol., 182:626-646 (1990); Rattan, Ann. NY Acad. Sci., 663:48-62 (1992).) In some embodiments, the peptides are acetylated at the N-terminus and/or amidated at the C-terminus.

Any of numerous chemical modifications may be carried out by known techniques, including, but not limited to acetylation, formylation, etc. Additionally, derivatives may contain one or more non-classical amino acids.

In some embodiments, cyclization, or macrocyclization of the peptide backbone is achieved by sidechain-to-sidechain linkage formation. Methods for achieving this are well known in the art and may involve natural as well as unnatural amino acids. Approaches includes disulfide formation, lanthionine formation or thiol alkylations (e.g. Michael addition), amidation between amino and carboxylate sidechains, click chemistry (e.g. azide—alkyne condensation), peptide stapling, ring closing metathesis and the use of enzymes.

### Affinity Agents for Purification

In purification based on affinity chromatography, a target of interest (e.g. protein or molecule) is selectively isolated according to its ability to specifically and reversibly bind to a ligand that has typically been covalently coupled to a chromatographic matrix. The affinity ligands of the disclosure can be used as reagents for affinity purification of AAV8 or variants of AAV8 from clarified cell culture fluids (CCCF) or natural sources such as biological samples (e.g., serum), for example.

In some embodiments, a ligand or multimer that specifically binds AAV8 or a variant of AAV8 is immobilized on beads, such as agarose beads, to form an affinity separation matrix, and then used to affinity purify the target.

Methods of covalently coupling proteins to a surface are known by those of skill in the art, and peptide tags that can be used to attach a ligand to a solid surface are known to those of skill in the art. Further, ligands can be attached (i.e., coupled, linked, or adhered) to a solid surface using any reagents or techniques known in the art. In some embodiments, a solid support comprises beads, glass, slides, chips and/or gelatin. Thus, a series of ligands can be used to make an array on a solid surface using techniques known in the art. For example, U.S. Publ. No. 2004/0009530, which is incorporated herein by reference, discloses methods for preparing arrays.

In some embodiments, a ligand or multimer is used to isolate AAV8 particles or capsids or variant AAV8 particles or capsids by affinity chromatography. In some embodiments, a ligand or multimer is immobilized on a solid support. The ligand or multimer can be immobilized on the solid support using techniques and reagents described herein or otherwise known in the art. Suitable solid supports are described herein or otherwise known in the art and in specific embodiments are suitable for packing a chromatography column. The affinity agent can be packed in columns of various sizes and operated at various linear velocities or immobilized affinity ligand can be contacted with a solution under conditions favorable to form a complex between the ligand and AAV8 capsid or AAV8 variant capsid. Non-binding materials can be washed away. Suitable wash conditions can readily be determined by one of skill in the art. Examples of suitable wash conditions are described in Shukla and Hinckley, Biotechnol Prog. 2008 September-October; 24(5):1115-21. doi: 10.1002/btpr.50.

In some embodiments, chromatography is carried out by mixing a solution containing the target of interest and the ligand, then isolating complexes of the target of interest and ligand, e.g., AAV8 and ligand. For example, a ligand or multimer is immobilized on a solid support such as beads, then separated from a solution along with the AAV8 capsid or AAV8 variant capsid by filtration. In some embodiments, the ligand or multimer is a fusion protein that contains a peptide tag, such as a poly-His tail or streptavidin binding region, which can be used to isolate the ligand or multimer after complexes have formed using an immobilized metal affinity chromatographic resin or streptavidin-coated substrate. Once separated, the AAV8 or AAV8 variant capsid can be released from the ligand or multimer under elution conditions and recovered in a purified form.

In some embodiments, a ligand or multimer of the disclosure is coupled to a highly cross-linked agarose base matrix which is useful for bioprocess applications. Attachment of the ligand or multimer to the base matrix may be through a flexible spacer that ensures ligand accessibility and subsequently leads to high binding capacities. The affinity of the ligands and multimers of the disclosure ensures highly specific binding of AAV8 at near neutral pH (pH 6-9), while enabling elution at pH as high as 4.5. Moreover, the ligands of the disclosure are designed for enhanced alkali stability, enabling the repeated use of 0.5 M NaOH in cleaning-in-place and sanitization applications.

In another aspect, the disclosure provides, a method of isolating AAV8 particles or capsids and/or AAV8 variants particles or capsids, wherein a separation matrix as disclosed above is used. In certain embodiments, the method comprises the steps of (a) contacting a liquid sample comprising AAV8 particles and/or capsids and/or AAV8 variant particles and/or capsids with a separation matrix as disclosed above, (b) washing the separation matrix with a washing liquid, (c) eluting the AAV8 and or AAV8 variant particles and/or capsids from the separation matrix with an elution liquid, and (d) cleaning the separation matrix with a cleaning liquid, which can alternatively be called a cleaning-in-place (CIP) liquid, e.g. with a contact (incubation) time of at least one minute, e.g., for one to four minutes or more.

Suitable compositions of the liquid sample, the washing liquid and the elution liquid, as well as the general conditions for performing the separation are well known in the art of affinity chromatography. A liquid sample comprising AAV8 and/or AAV8 variant particles and/or capsids may comprise host cell proteins (HCP), such as HEK293T cells for example. The host cell proteins may be desorbed during step (b).

Binding of AAV8 has been demonstrated with buffers at near-neutral pH (6-9) over a wide range of ionic strength (e.g., 100-400 mM NaCl). Conventional buffers, e.g., phosphate, citrate, acetate, Tris, may be used for equilibration and loading.

In some embodiments, a solution or sample containing AAV8 particles or capsids and/or AAV8 variant particles or capsids (i.e., virus particles/capsids) is concentrated, for example by ultrafiltration, prior to contacting the solution with the separation matrix. For example, the viral particle/capsid-containing solution, e.g., a clarified cell culture feed, may be concentrated up to 20-fold. Concentration of the virus particles/capsids reduces the load time for affinity chromatography. Increase in concentration may also have a positive effect on the binding capacity due to thermodynamic equilibrium effects, which may lead to a lower volume of separation matrix needed for purification. Concentrating the feed of viral/capsid solution can also lead to a significant gain in the processing time.

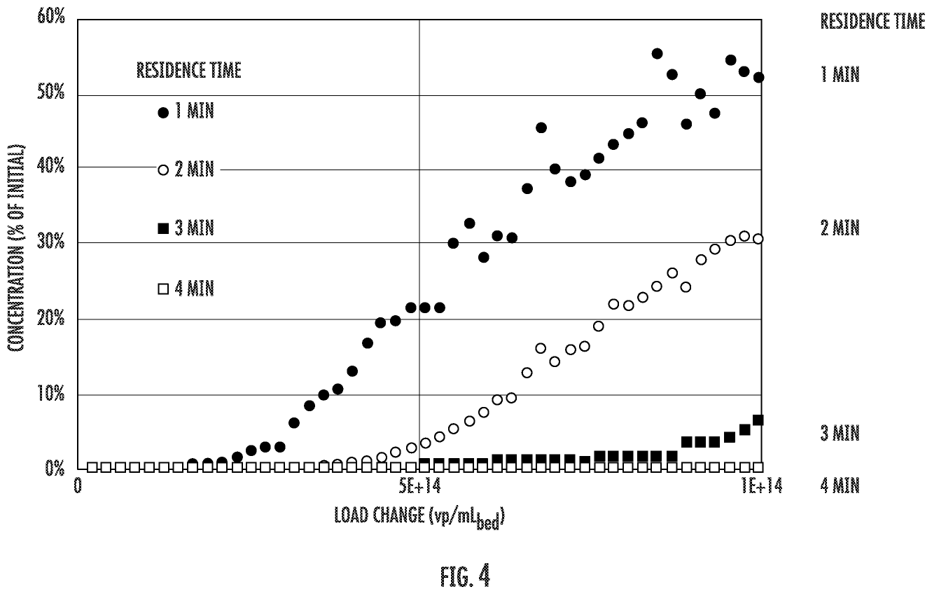

Alternatively, the solution or sample containing AAV8 particles or capsids and/or AAV8 variant particles or capsids is a non-concentrated or diluted solution, e.g., a clarified cell culture feed (CCCF). As shown in FIG. 4, the affinity separation matrix of the disclosure is characterized by an ability to process CCCF at high volumetric flow rates, enabling capture from dilute CCCF feed streams.

Elution of viral particles and capsids is generally achieved by lowering the pH, e.g., 2.0-3.0, although higher pH may be used. Optimal conditions for elution of AAV8 and variants thereof can be readily determined by those of skill in this field.

The affinity agents of the disclosure are alkali-tolerant, enabling the use of NaOH up to concentrations of 0.5 M for cleaning. In certain embodiments, a CIP regimen of 0.5 M NaOH exposure for up to 30 to 60 minutes per cycle, for example, ensures consistent chromatographic performance for several cycles, e.g., 15-30 cycles, including up to 70%-90% of the initial AAV8 binding capacity and low residual DNA and HCP levels, as well as substantially no change in flow capacity.

### EXAMPLES

### Example 1

Peptides were synthesized by standard Fmoc solid phase peptide synthesis techniques and purified by preparative reverse phase UPLC. The purity of peptides was assessed by RP HPLC with both UV and quadrupole time-of-flight mass spectrometric detection.

Recombinant protein ligands were expressed in E. coli and/or Pichia pastoris using standard techniques. Ligands were purified using multi-column chromatography. For his-tagged ligands IMAC was used as the primary capture step. Biotinylated ligands were generated with the Avitag™ system (Avidity, Aurora, CO). Non-biotinylated ligands bearing the Avitag™ sequence were prepared by omitting exogenous biotin. The purity and identity of recombinant protein ligands was assessed by a combination of SDS-PAGE, RP UPLC, quadrupole time-of-flight mass spectrometry and SEC (Sephadex S75, Cytiva, Marlborough, MA). In many instances the ligand is isolated without the N-terminal methionine residue, which is presumed to be cleaved during expression. In many instances a mixture is obtained where only a portion of the purified ligand contains an N-terminal methionine. The presence or absence of an N-terminal methionine does not affect the binding specificity or other properties of the ligands.

### Example 2

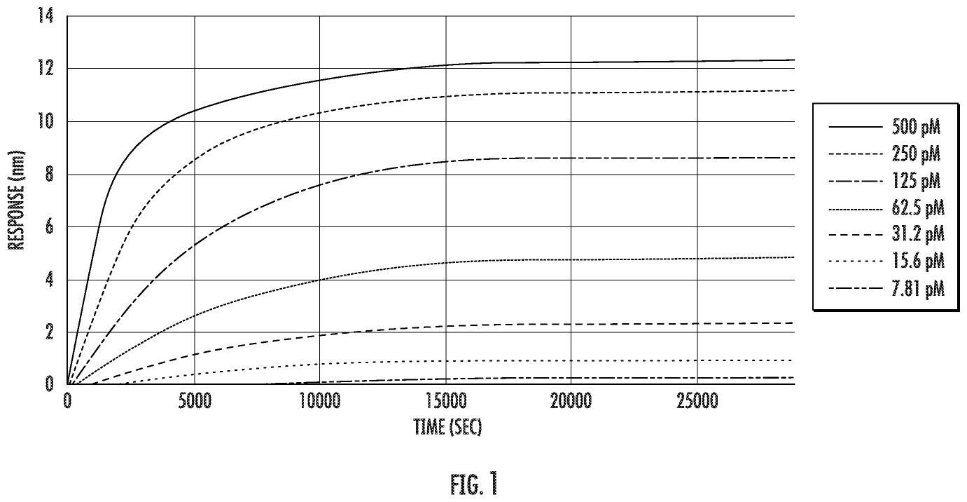

This example demonstrates the binding of biotinylated ligands to AAV8 capsids using biolayer interferometry (ForteBio, Menlo Park, CA. Biotinylated ligands, were immobilized on sensors and incubated with solutions containing 5×1011 vp/mL in 100 mM sodium phosphate, 100 mM sodium chloride, 0.01% (w/v) bovine serum albumin and 0.1% (v/v) Triton X-100, pH 7.0. A blank sensor and a non-binding sequence (SEQ ID No. 54) were included as controls. The association phase showed the initial linear increase in response that it is typical for AAV. As the sensor became saturated the sensorgram showed greater curvature. An example is shown in FIG. 1 for ligand SEQ ID No. 14. The response was measured after 4000 seconds incubation time and are listed below.

| Seq ID NO: | Response |
|---|---|
| 14 | 7.31 |
| 15 | 4.47 |
| 16 | 6.48 |
| 17 | 5.99 |
| 18 | 6.19 |
| 19 | 6.29 |
| 20 | 5.56 |
| 21 | 6.00 |
| 22 | 6.16 |
| 23 | 6.21 |
| 24 | 5.91 |
| 25 | 6.23 |
| 26 | 4.94 |
| 27 | 4.47 |
| 28 | 6.06 |
| 29 | 5.85 |
| 30 | 6.18 |
| 31 | 5.79 |
| 32 | 4.95 |
| 33 | 5.60 |
| 34 | 5.56 |
| 35 | 3.95 |
| 36 | 5.48 |
| 37 | 5.67 |
| 38 | 5.54 |
| 39 | 5.75 |
| 40 | 5.98 |
| 41 | 6.34 |
| 42 | 6.23 |
| 43 | 6.02 |
| 44 | 5.56 |
| 45 | 5.92 |
| 46 | 5.96 |
| 47 | 5.82 |
| 48 | 6.20 |
| 49 | 5.08 |
| 50 | 5.79 |
| 51 | 5.50 |
| 52 | 5.79 |
| Blank sensor | 0.05 |
| 94 | 0.03 |

### Example 3

This example demonstrates the sodium hydroxide stability of the affinity ligands. Ligands were incubated in 0.5 M NaOH for 16 hours and then neutralized. The binding of the NaOH treated ligands was measured as described in Example 1 and compared to untreated ligand. The binding retained was calculated according to the following formula:

% binding retained=(measured response after NaOH treatment)÷(measured response of untreated)×100

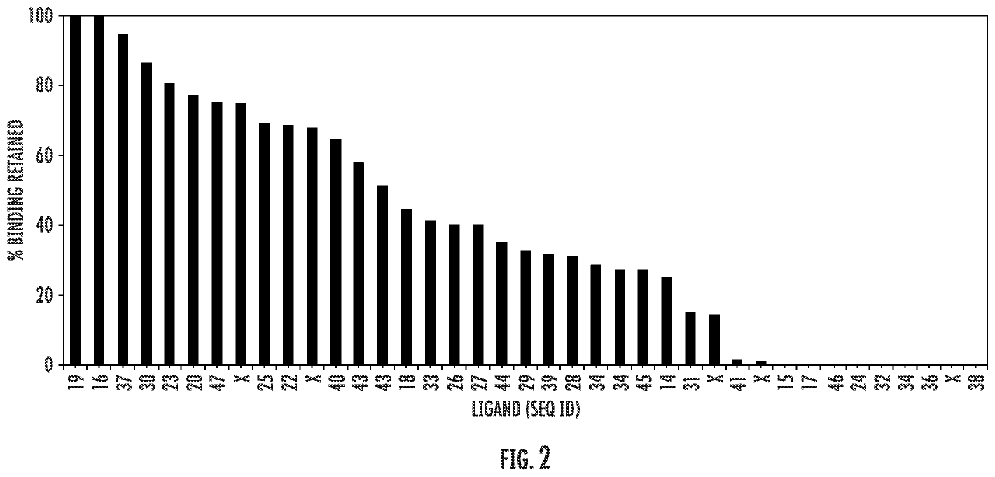

The data are shown in FIG. 2, which shows that many of the affinity ligands of the disclosure exhibit high stability under tested conditions.

### Example 4

This example demonstrates use of affinity agents comprising affinity ligands described herein for affinity purification of AAV8 particles. Clarified cell culture feed stream (CCCF) containing AAV8 viral capsids at a titer of approximately 8E12 total capsids/mL was used. A 0.3 cm ID×5 cm column was operated as shown in the following table. Resins comprised a ligand density of 1.9-2.0 mg/mL.

TABLE 1

| | | | Linear |
|---|---|---|---|
| | | Volume | Velocity |
| Step | Solution ID | (CV) | (cm/hr) |
| Equilibration | 20 mM Tris-HCl, 400 mM NaCl, | 2.8 | 75 |
| | pH 7.5 | | |
| Load Inject | CCCF - 8E12 vp/mL | 65 | 75 |
| Wash | 20 mM Tris-HCl, 400 mM NaCl, | 28 | 75 |
| (optional) | pH 7.5 | | |
| Elution | 50 mM glycine, 150 mM NaCl, | 28 | 50 |
| | pH 3.0 | | |
| Strip | 0.1M NaOH | 28 | 75 |

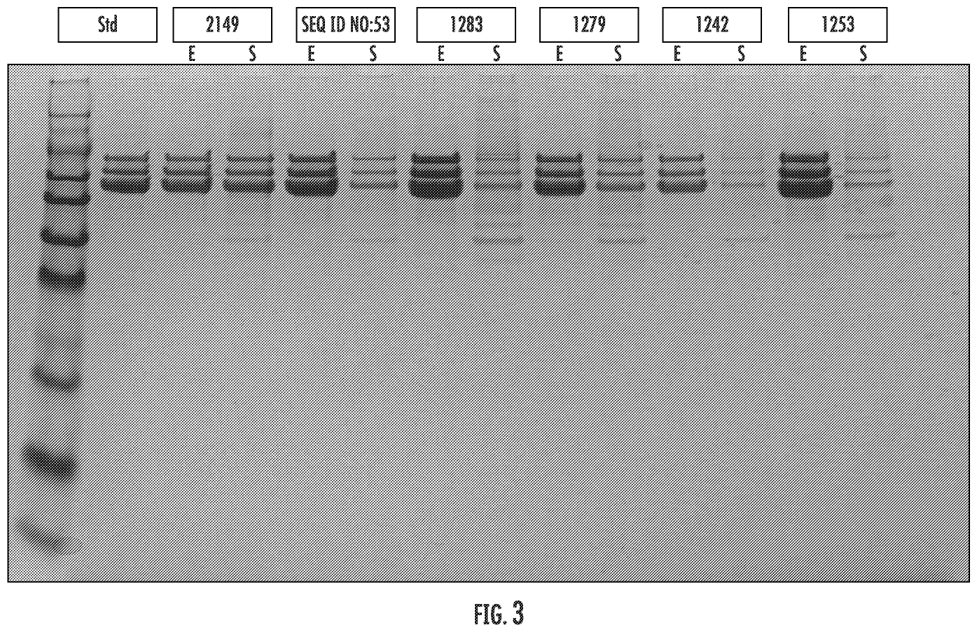

The eluted materials were analyzed by SDS-PAGE alongside the strip fractions. The data are shown in FIG. 3. The radiograph shows that the affinity resin comprising the affinity ligand having an amino acid sequence of SEQ ID NO. 53 results in high yield and purity of AAV8 capsids in the eluate.

### Example 5

This example demonstrates that the affinity agents of the disclosure enable mild elution conditions. A 3 mm ID×25 mm column packed with resin comprising ligand SEQ ID No. 14 was challenged with −3.5E14 vp/mL of resin and eluted with pH 4 buffer containing either 1,6-hexanediol or propylene glycol. The results shown in the following table indicate that the use of these additives enables elution at higher pH. Hexanediol afforded higher yield and better purity in this experiment. HCP=host cell protein.

TABLE 2

| Additive | 20% 1,6-Hexanediol | 70% Propylene glycol |
|---|---|---|
| Yield (vp/mL resin) | 2.9E14 | 1.5E14 |
| HCP (ppm) | 1900 | 3800 |
| DNA (ppm) | 540 | 430 |

### Example 6

This example demonstrates the high binding capacity of the disclosed affinity agents and demonstrates that high binding capacities are maintained at faster flow rates (i.e., shorter residence times).

An affinity agent comprising an affinity ligand corresponding to SEQ ID NO. 53 bound to a resin was packed into a 3 mm×50 mm column, which was challenged with purified AAV8 capsids. The breakthrough curves shown in FIG. 5 were obtained at 1, 2, 3, and 4 minute residence times. Capsids in the flow-through were measured by capsid ELISA. The high capacity binding at short residence times yields very high productivities, enabling the use of the affinity agent for purification of AAV8 capsids at various process configurations and scales.

The above examples demonstrate that the affinity resins of the disclosure can be fine-tuned to achieve different performance features that different applications may require. Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

Any methods disclosed herein need not be performed in the order recited. The methods disclosed herein include certain actions taken by a practitioner; however, they can also include any third-party instruction of those actions, either expressly or by implication.

TABLE 3
SEQUENCES

| SEQ ID | |
|---|---|
| NO | SEQUENCE |
| 14 | MAQGTVDAKFDKELEEARAEIERLPNLTERQRRSFIYRLRQDPSFSAILLADAK |
| | YRNDIQAPKGQAGQGGGSGLNDIFEAQKIEWHEHHHHHH |
| 15 | MAQGTVDAKFDKELEEARAEIERLPNLTERQRRSFIYRLRQDPSFSAHLLADAK |
| | YRNDTQAPKGQAGQGGGSGLNDIFEAQKIEWHEHHHHHH |
| 16 | MAQGTVDAKFDKELEEARAEIERLPNLTENQRRSFIYRLRQDPSFSAILLADAK |
| | YRNDIQAPKGQAGQGGGSGLNDIFEAQKIEWHEHHHHHH |
| 17 | MAQGTVDAKFDKELEEARAEIERLPNLTERQRRSFIYRLRQDPSPSAILLADAK |
| | YRNDIQAPKGQAGQGGGSGLNDIFEAQKIEWHEHHHHHH |
| 18 | MAQGTVDAKFDKELEEARAEIERLPNLTERQRRSFIYRLRQDPSVSAILLADAK |
| | YRNDIQAPKGQAGQGGGSGLNDIFEAQKIEWHEHHHHHH |
| 19 | MAQGTVDAKFDKELEEARAEIERLPNLTESQRRSFIYRLRQDPSFSAILLADAK |
| | YRNDIQAPKGQAGQGGGSGLNDIFEAQKIEWHEHHHHHH |
| 20 | MAQGTVDAKFDKELEEARAEIERLPNLTERQRRSFIYRLRQDPSYSAILLADAK |
| | YRNDIQAPKGQAGQGGGSGLNDIFEAQKIEWHEHHHHHH |
| 21 | MAQGTVDAKFDKELEEARAEIERLPNLTERQRRSFIYRLREDPSFSAILLADAK |
| | YRNDSQAPKGQAGQGGGSGLNDIFEAQKIEWHEHHHHHH |
| 22 | MAQGTVDAKFDKELEEARAEIERLPNLTERQRRSFIYRLRQDPSFSAILLADAK |
| | YRNDQQAPKGQAGQGGGSGLNDIFEAQKIEWHEHHHHHH |
| 23 | MAQGTVDAKFDKELEEARAEIERLPNLTERQRRSFIYRLRQDPSFSAILLADAK |
| | YRNDAQAPKGQAGQGGGSGLNDIFEAQKIEWHEHHHHHH |
| 24 | MAQGTVDAKFDKELEEARAEIERLPNLTEAQRRSFIYRLRQDPSFSAILLADAK |
| | YRNDFQAPKGQAGQGGGSGLNDIFEAQKIEWHEHHHHHH |
| 25 | MAQGTVDAKFDKELEEARAEIERLPNLTEDQRRSFIYRLRQDPSFSAILLADAK |
| | YRNDIQAPKGQAGQGGGSGLNDIFEAQKIEWHEHHHHHH |
| 26 | MAQGTVDAKFDKELEEARAEIERLPNLTERQRRPFIYRLRQDPSFSAILLADAK |
| | YRNDIQAPKGQAGQGGGSGLNDIFEAQKIEWHEHHHHHH |
| 27 | MAQGTVDAKFDKELEEARAEIERLPNLTERQRRSFIYRLRQDPSFSAILLADAK |
| | YLNDIQAPKGQAGQGGGSGLNDIFEAQKIEWHEHHHHHH |
| 28 | MAQGTVDAKFDKELEEARAEIERLPNLTERQRRSFIYRLRQDPSFSAILLADAK |
| | YRNDDQAPKGQAGQGGGSGLNDIFEAQKIEWHEHHHHHH |
| 29 | MAQGTVDAKFDKELEEARAEIERLPNLTERQRRSFIYRLRQDPSFSAILLADAK |
| | YRNDRQAPKGQAGQGGGSGLNDIFEAQKIEWHEHHHHHH |
| 30 | MAQGTVDAKFDKELEEARAEIERLPNLTELQRRSFIYRLRQDPSFSAILLADAK |
| | YRNDIQAPKGQAGQGGGSGLNDIFEAQKIEWHEHHHHHH |
| 31 | MAQGTVDAKFDKELEEARAEIERLPNLTERQRRSFIYRLRQDPSFSAILLADAK |
| | YRNDGQAPKGQAGQGGGSGLNDIFEAQKIEWHEHHHHHH |
| 32 | MAQGTVDAKFDKELEEARAEIERLPNLTERQRRSFIYRLRQDPSFSAILLADAK |
| | TRNDIQAPKGQAGQGGGSGLNDIFEAQKIEWHEHHHHHH |
| 33 | MAQGTVDAKFDKELEEARAEIERLPNLTERQRRSFIYRLRQDPSFSAILLADAK |
| | YRNDKQAPKGQAGQGGGSGLNDIFEAQKIEWHEHHHHHH |
| 34 | MAQGTVDAKFDKELEEARAEIERLPNLTERQRRSFIYRLRQDPSFSAILLADAK |
| | YRNDYQAPKGQAGQGGGSGLNDIFEAQKIEWHEHHHHHH |
| 35 | MAQGTVDAKFDKELEEARAEIERLPNLTERQRRSFIYRLRQDPSFSAILLADAK |
| | YRNDPQAPKGQAGQGGGSGLNDIFEAQKIEWHEHHHHHH |
| 36 | MAQGTVDAKFDKELEEARAEIERLPNLTERQRRSFIYRLRQDPSFSANLLADAK |
| | YRNDEQAPKGQAGQGGGSGLNDIFEAQKIEWHEHHHHHH |
| 37 | MAQGTVDAKFDKELEEARAEIERLPNLTERQRRHFIYRLRQDPSFSAILLADAK |
| | YRNDIQAPKGQAGQGGGSGLNDIFEAQKIEWHEHHHHHH |
| 38 | MAQGTVDAKFDKELEEARAEIERLPNLTERQRRSFIYRLRQDPSFSAILLADAK |
| | QRNDIQAPKGQAGQGGGSGLNDIFEAQKIEWHEHHHHHH |
| 39 | MAQGTVDAKFDKELEEARAEIERLPNLTERQRRSFIYSLRQDPSFSAILLADAK |
| | YRNDIQAPKGQAGQGGGSGLNDIFEAQKIEWHEHHHHHH |
| 40 | MAQGTVDAKFDKELEEARAEIERLPNLTERQRRSFIARLRQDPSFSAILLADAK |
| | YRNDIQAPKGQAGQGGGSGLNDIFEAQKIEWHEHHHHHH |
| 41 | MAQGTVDAKFDKELEEARAEIERLPNLTERQRRSFIYRLRQDPSFSAILLADAK |
| | YRNDSQAPKGQAGQGGGSGLNDIFEAQKIEWHEHHHHHH |
| 42 | MAQGTVDAKFDKELEEARAEIERLPNLTERQRRSFIYRLRQDPSFSAILLADAK |
| | YRNDVQAPKGQAGQGGGSGLNDIFEAQKIEWHEHHHHHH |
| 43 | MAQGTVDAKFDKELEEARAEIERLPNLTEIQRRSFIYRLRQDPSFSAILLADAKY |
| | RNDIQAPKGQAGQGGGSGLNDIFEAQKIEWHEHHHHHH |
| 44 | MAQGTVDAKFDKELEEARAEIERLPNLTERQRRSFIYRLRHDPSFSAILLADAK |
| | YRNDRQAPKGQAGQGGGSGLNDIFEAQKIEWHEHHHHHH |
| 45 | MAQGTVDAKFDKELEEARAEIERLPNLTERQRRSFIYRLRQDPSFSAILLADAK |
| | YRNDTQAPKGQAGQGGGSGLNDIFEAQKIEWHEHHHHHH |
| 46 | MAQGTVDAKFDKELEEARAEIERLPNLTERQRRSFIYRLRQDPSPSAILLADAK |
| | YRNDLQAPKGQAGQGGGSGLNDIFEAQKIEWHEHHHHHH |
| 47 | MAQGTVDAKFDKELEEARAEIERLPNLTERQRRSFIYRLRQDPSFSAILLADAK |
| | YRNDLQAPKGQAGQGGGSGLNDIFEAQKIEWHEHHHHHH |
| 48 | MAQGTVDAKFDKELEEARAEIERLPNLTERQRRSFIYRLRQDPEFSAILLADAK |
| | YRNDIQAPKGQAGQGGGSGLNDIFEAQKIEWHEHHHHHH |
| 49 | MAQGTVDAKFDKELEEARAEIERLPNLTERQRRSFIYRLRQDPSFSRILLEDAKY |
| | RNDIQAPKGQAGQGGGSGLNDIFEAQKIEWHEHHHHHH |
| 50 | MAQGTVDAKFDKELEEARAEIERLPNLTERQRRSFIYRLRQDPSFSEILLRDAKY |
| | RNDIQAPKGQAGQGGGSGLNDIFEAQKIEWHEHHHHHH |
| 51 | MAQGTVDAKFDKELEEARAEIERLPNLTERQRRSFIYRLRQDPSFSAILLADAK |
| | YRADIQAPKGQAGQGGGSGLNDIFEAQKIEWHEHHHHHH |
| 52 | MAQGTVDAKFDKELEEARAEIERLPNLTERQRRSFIYRLRQDPSFSAILLADAK |
| | YRNRIQAPKGQAGQGGGSGLNDIFEAQKIEWHEHHHHHH |
| 53 | MVDAKFDKELEEARAEIERLPNLTERQRRSFIYRLRQDPSFSAILLADAKYRNDI |
| | QAPKVD |
| 54 | VDAKFDKELEEARAEIERLPNLTERQRRSFIYRLRQDPSFSAILLADAKYRNDIQ |
| | APK |
| 55 | VDAKFDKELEEARAEIERLPNLTERQRRSFIYRLRQDPSFSAHLLADAKYRNDT |
| | QAPK |
| 56 | VDAKFDKELEEARAEIERLPNLTENQRRSFIYRLRQDPSFSAILLADAKYRNDIQ |
| | APK |
| 57 | VDAKFDKELEEARAEIERLPNLTERQRRSFIYRLRQDPSPSAILLADAKYRNDIQ |
| | APK |
| 58 | VDAKFDKELEEARAEIERLPNLTERQRRSFIYRLRQDPSVSAILLADAKYRNDIQ |
| | APK |
| 50 | VDAKFDKELEEARAEIERLPNLTESQRRSFIYRLRQDPSFSAILLADAKYRNDIQ |
| | APK |
| 60 | VDAKFDKELEEARAEIERLPNLTERQRRSFIYRLRQDPSYSAILLADAKYRNDIQ |
| | APK |
| 61 | VDAKFDKELEEARAEIERLPNLTERQRRSFIYRLREDPSFSAILLADAKYRNDSQ |
| | APK |
| 62 | VDAKFDKELEEARAEIERLPNLTERQRRSFIYRLRQDPSFSAILLADAKYRNDQ |
| | QAPK |
| 63 | VDAKFDKELEEARAEIERLPNLTERQRRSFIYRLRQDPSFSAILLADAKYRNDA |
| | QAPK |
| 64 | VDAKFDKELEEARAEIERLPNLTEAQRRSFIYRLRQDPSFSAILLADAKYRNDFQ |
| | APK |
| 65 | VDAKFDKELEEARAEIERLPNLTEDQRRSFIYRLRQDPSFSAILLADAKYRNDIQ |
| | APK |
| 66 | VDAKFDKELEEARAEIERLPNLTERQRRPFIYRLRQDPSFSAILLADAKYRNDIQ |
| | APK |
| 67 | VDAKFDKELEEARAEIERLPNLTERQRRSFIYRLRQDPSFSAILLADAKYLNDIQ |
| | APK |
| 68 | VDAKFDKELEEARAEIERLPNLTERQRRSFIYRLRQDPSFSAILLADAKYRNDD |
| | QAPK |
| 69 | VDAKFDKELEEARAEIERLPNLTERQRRSFIYRLRQDPSFSAILLADAKYRNDRQ |
| | APK |
| 70 | VDAKFDKELEEARAEIERLPNLTELQRRSFIYRLRQDPSFSAILLADAKYRNDIQ |
| | APK |
| 71 | VDAKFDKELEEARAEIERLPNLTERQRRSFIYRLRQDPSFSAILLADAKYRNDG |
| | QAPK |
| 72 | VDAKFDKELEEARAEIERLPNLTERQRRSFIYRLRQDPSFSAILLADAKTRNDIQ |
| | APK |
| 73 | VDAKFDKELEEARAEIERLPNLTERQRRSFIYRLRQDPSFSAILLADAKYRNDK |
| | QAPK |
| 74 | VDAKFDKELEEARAEIERLPNLTERQRRSFIYRLRQDPSFSAILLADAKYRNDY |
| | QAPK |
| 75 | DAKFDKELEEARAEIERLPNLTERQRRSFIYRLRQDPSFSAILLADAKYRNDPQA |
| | PK |
| 76 | DAKFDKELEEARAEIERLPNLTERQRRSFIYRLRQDPSFSANLLADAKYRNDEQ |
| | APK |
| 77 | VDAKFDKELEEARAEIERLPNLTERQRRHFIYRLRQDPSFSAILLADAKYRNDIQ |
| | APK |
| 78 | VDAKFDKELEEARAEIERLPNLTERQRRSFIYRLRQDPSFSAILLADAKQRNDIQ |
| | APK |
| 79 | VDAKFDKELEEARAEIERLPNLTERQRRSFIYSLRQDPSFSAILLADAKYRNDIQ |
| | APK |
| 80 | VDAKFDKELEEARAEIERLPNLTERQRRSFIARLRQDPSFSAILLADAKYRNDIQ |
| | APK |
| 81 | VDAKFDKELEEARAEIERLPNLTERQRRSFIYRLRQDPSFSAILLADAKYRNDSQ |
| | APK |
| 82 | VDAKFDKELEEARAEIERLPNLTERQRRSFIYRLRQDPSFSAILLADAKYRNDV |
| | QAPK |
| 83 | VDAKFDKELEEARAEIERLPNLTEIQRRSFIYRLRQDPSFSAILLADAKYRNDIQ |
| | APK |
| 84 | VDAKFDKELEEARAEIERLPNLTERQRRSFIYRLRHDPSFSAILLADAKYRNDRQ |
| | APK |
| 85 | VDAKFDKELEEARAEIERLPNLTERQRRSFIYRLRHDPSFSAILLADAKYRNDRQ |
| | APK |
| 86 | VDAKFDKELEEARAEIERLPNLTERQRRSFIYRLRQDPSPSAILLADAKYRNDLQ |
| | APK |
| 87 | VDAKFDKELEEARAEIERLPNLTERQRRSFIYRLRQDPSFSAILLADAKYRNDLQ |
| | APK |
| 88 | VDAKFDKELEEARAEIERLPNLTERQRRSFIYRLRQDPEFSAILLADAKYRNDIQ |
| | APK |
| 89 | VDAKFDKELEEARAEIERLPNLTERQRRSFIYRLRQDPSFSRILLEDAKYRNDIQ |
| | APK |
| 90 | DAKFDKELEEARAEIERLPNLTERQRRSFIYRLRQDPSFSEILLRDAKYRNDIQA |
| | PK |
| 91 | VDAKFDKELEEARAEIERLPNLTERQRRSFIYRLRQDPSFSAILLADAKYRADIQ |
| | APK |
| 92 | VDAKFDKELEEARAEIERLPNLTERQRRSFIYRLRQDPSFSAILLADAKYRNRIQ |
| | APK |
| 93 | VDAKFDKELEEARAEIERLPNLTERQRRSFIYRLRQDPSFSAILLADAKYRNDIQ |
| | APK |
| 94 | MAQGTVDAKFDKEQIEADTEIIWLPNLNLLQFKAFIKSLLDDPSQSANLLAEAK |
| | KLNDAQAPKGQAGQGGGSGLNDIFEAQKIEWHEHHHHHH |

1. An affinity ligand comprising an amino acid sequence represented by the formula, from N-terminus to C-terminus,
(SEQ ID NO: 1)
[A]-X₁QRRX₂FIX₃X₄LRX₅DPX₆X₇SX₈X₉LLX₁₀
X₁₁AX₁₂X₁₃X₁₄X₁₅X₁₆X₁₇-[B]
wherein
(a) [A] comprises a first α-helix-forming peptide domain;
(b) X₁ is A, R, N, S, D, L, Q or I, preferably R;
(c) X₂ is G, H, P or S, preferably S;
(d) X₃ is A or Y, preferably Y;
(e) X₄ is R or S, preferably R;
(f) X₅ is E, H or Q, preferably Q;
(g) X₆ is E or S, preferably S;
(h) X₇ is F, V or Y, preferably F;
(i) X₈ is A, E or R, preferably A;
(j) X₉ is H, I or N, preferably H;
(k) X₁₀ is A, E or R, preferably A;
(l) X₁₁ is D or E, preferably D;
(m) X₁₂ is K or R, preferably K;
(n) X₁₃ is Q, T or Y, preferably Y;
(o) X₁₄ is D, L or R, preferably R;
(p) X₁₅ is A or N, preferably N;
(q) X₁₆ is D, L or R, preferably R;
(r) X₁₇ is A, D, E, F, G, I, K, L, P, Q, R, S, T or Y, preferably I;
(s) [B] is comprises a peptide comprising an amino acid sequence of QAPX₁₈ (SEQ ID NO: 2) or QAPX₁₈VD (SEQ ID NO: 3), wherein X₁₈ is A, K or R; and
wherein said affinity ligand specifically interacts with an adeno-associated virus subtype 8 (AAV8) particle or capsid or a variant of an AAV8 particle or capsid.

2. The affinity agent of claim 1, wherein [A] comprises a peptide having an amino acid sequence of SEQ ID NO: 4.

3. The affinity agent of claim 1, wherein [A] comprises a peptide having an amino acid sequence of SEQ ID NO: 5.

4. The affinity ligand of claim 1, wherein the N-terminus of [A] is M or MAQGT (SEQ ID NO: 6).

5. The affinity ligand of claim 1, wherein [B] has an amino acid sequence of QAPKVD (SEQ ID NO: 7) or QAPRVD (SEQ ID NO: 8).

6. The affinity ligand of claim 1, wherein [B] comprises a peptide having an amino acid sequence of QAPX₁₈-[C] (SEQ ID NO: 9), wherein [C] is a peptide domain selected from the group consisting of

| (SEQ ID NO: 10) | |
|---|---|
| (a) | VDGQAGQGGGSGLNDIFEAQKIEWHEHHHHHH, |
| (SEQ ID NO:11) | |
| (b) | GQAGQGGGSGLNDIFEAQKIEWHEHHHHHH, |
| (SEQ ID NO: 12) | |
| (c) | VDGLNDIFEAQKIEWHEHHHHHH, |
| and | |
| (SEQ ID NO: 13) | |
| (d) | GLNDIFEAQKIEWHEHHHHHH. |

7. The affinity ligand of claim 1, which further comprises a C-terminal cysteine or lysine.

8. The affinity ligand of claim 1, wherein said ligand comprises any one of SEQ ID Nos: 14-93.

9. (canceled)

10. The affinity ligand of claim 1, wherein said ligand further comprises at least one heterologous agent operably linked to said affinity ligand to thereby form a conjugate;
wherein said heterologous agent is selected from the group consisting of one or more small molecule diagnostic or therapeutic agents; a DNA, RNA, or hybrid DNA-RNA molecule; traceable marker; radioactive agent an antibody; a single chain variable domain; and an immunoglobulin fragment.

11. (canceled)

12. A multimer comprising a plurality of affinity ligands according to claim 1;
wherein the multimer is a dimer, trimer, tetramer, pentamer, hexamer, heptamer, octamer, or nonamer.

13. (canceled)

14. A nucleic acid or vector encoding an affinity ligand of claim 1.

15. A separation matrix, comprising at least one affinity ligand of claim 1.

16. The separation matrix of claim 15, wherein the at least one affinity ligand is coupled to a solid support.

17. The separation matrix of claim 16, wherein the affinity ligands or multimers are coupled to the solid support via carbamate bonds.

18. The separation matrix of claim 16, wherein the solid support is a chromatographic resin or matrix.

19. The separation matrix of claim 18, wherein the solid support is a cross-linked agarose matrix.

20. A method of isolating adeno-associated virus subtype 8 (AAV8) particles or capsids, comprising contacting AAV8 particles or capsids with a separation matrix of claim 15.

21. The method of claim 20, comprising the steps of (a) contacting a liquid sample comprising AAV8 particles or capsids with the separation matrix, (b) washing said separation matrix with a washing liquid, (c) eluting the AAV8 particles or capsids from the separation matrix with an elution liquid, and (d) cleaning the separation matrix with a cleaning liquid.

22. The method of claim 21, wherein the cleaning liquid comprises 0.1-0.5 M NaOH.

23. The method of claim 21, wherein steps (a)-(d) are repeated at least 10 times.

24. An expression system, comprising the nucleic acid or vector of claim 14.
